(19) United States
(12) Patent Application Publication
(10) Pub. No.: US 2005/0002935 A1
(43) Pub. Date: Jan. 6, 2005

(54) Use of B7-H3 as an immunoregulatory agent

(76) Inventors: Vincent Ling, Walpole, MA (US); Beatriz Carreno, Acton, MA (US); Mary Collins, Natick, MA (US)

Correspondence Address: Finnegan, Henderson, Farabow,;Garrett & Dunner, L.L.P., 1300 I Street, N.W., Washington, DC, 20005-3315, US

(21) Appl. No.: 10/824,481

(22) Filed: Apr. 15, 2004

Related U.S. Application Data
(60) Provisional application No. 60/463,342, filed on Apr. 17, 2003.

(51) Int. Cl. A61K 38/17; A61K 39/395

(52) U.S. Cl. 424/145.1; 514/12

(57) ABSTRACT

The present disclosure relates to the fields of immunology and clinical immunology, and more particularly to the use of B7-family ligands and agonists and antagonists thereof in modulation of immune responses. The invention provides methods for modulation of lymphocyte activation involving the use of B7-H3, including B7-H3 VC and B7-H3 VCVC, and related molecules such as, for example, antibodies and nucleic acids.

### DESCRIPTION OF THE INVENTION

1. Field of the Invention

The present disclosure relates to the fields of immunology and clinical immunology, and more particularly to the use of B7-family ligands and agonists and antagonists thereof in modulation of immune responses.

2. Background of the Invention

Lymphocyte activation is a multi-step process requiring several signaling events between the lymphocyte and an “accessory” cell—an antigen presenting cell (APC) in the case of T cell activation, or a helper T cell for B cell activation). For lymphocyte activation to occur, two types of signals must be delivered to a resting lymphocyte. The primary type (stimulatory) confers specificity to the immune response and is mediated by the antigen-specific receptor (TcR—on T cells, and BcR—on cells) upon recognition of the antigenic peptide-MHC complex. The second type (costimulatory) is responsible for the magnitude of the response. This signal is mediated via “accessory” receptors expressed on the surface of the lymphocyte. The requirement for costimulation allows lymphocyte activation to be strictly regulated. Costimulation is in turn regulated by the inhibitory receptors on these cells that can deliver negative signals which counteract the positive costimulatory signals.

Mounting evidence suggests that a large number of structurally related ligands and receptors belonging to the immunoglobulin (Ig) superfamily of molecules interact and balance signals used during the process of lymphocyte activation (Frauwirth et al. (2002) J. Clin. Invest., 109:295-299). One such structurally related group includes ligands of the B7 family (B7-1, B7-2, ICOS-L, PD-L1, PD-L2, and B7-H3), which share similar domain structure having one Ig-V-like (“V”) domain and one Ig-C-like (“C”) domain which reside in the extracellular portion of the molecule (Sharpe et al. (2002) Nature Rev. Immunol., 2:116-126). In exon deletion and crystallographic studies of B7 proteins, it has been demonstrated that receptor-ligand interaction occurs via the V domains (Ostrov et al. (2000) Science, 290:816-819), while the C domains serve as structural support means for the V domains.

B7-1 and B7-2 can deliver either positive signals (through their low affinity cognate receptor CD28) or negative signals (through the high affinity receptor CTLA4). ICOS-L interaction with ICOS receptor results in positive signaling whereas PD-L1 delivers negative signals through its receptor, PD-1 (Carreno et al. (2002) Annual Rev. Immunol., 20:29-53).

The present disclosure relates to the modulation of immune responses regulated by the newest member of the B7 family of ligands, B7-H3. Human B7-H3 was originally identified as a B7-like protein which shares 20-27% amino acid identity with other B7 family members and has one V and one C domain (Chapoval et al. (2001) Nat. Immunol., 2:269-274). However, a more detailed analysis of partial B7-H3 EST clones demonstrated a variation in the gene exon structure in mammalian species (Sun et al. (2002) J. Immunol., 168:6294-6297). In particular, in primates, B7-H3 cDNA exists in two forms: one encodes a single set single set of V and C domains (“VC form”), and the other encodes a duplicated set of V and C domains (“VCVC form”). In contrast to the primate B3-H3, rodent B7-H3 cDNA exists only in a single form as VC.

B7-H3 VC has been initially characterized as a costimulatory ligand in both human (Chapoval et al. (2001) Nat. Immunol., 2:269-274 and U.S. Patent Application Pub. No. 2002/0168762) and mouse (Sun et al. (2002) J. Immunol., 168:6294-6297). In particular, it has been reported that costimulation of human T cells with B7-H3 VC results in enhanced T cell proliferation, induction of cytotoxic T cells, and increased gamma interferon transcript expression. Additionally, binding experiments in cell-based assays suggest that B7-H3 VC binds to a receptor expressed on activated T cells, which is not CTLA-4, ICOS, or PD-1.

In general, a need exists to provide therapeutic methods for immune system-related disorders and conditions. Appropriate modulation of immune responses can be accomplished by manipulation of the B7-H3 pathway.

### SUMMARY OF THE INVENTION

It is one of the objects of the present invention to provide methods and compositions for modulation of immune responses. Additional objects of the invention will be set forth in part in the following description and in part will be understood from the description or may be learned by practice of the invention.

The present invention is based, in part, on the discovery and demonstration that the VCVC form of B7-H3 accounts for the majority of B7-H3 transcripts seen across multiple tissues while the VC form of B7-H3 is only a minor transcript. The invention is further based, in part, on the discovery and demonstration that both forms of B7-H3, VC and VCVC, exhibit an inhibitory effect on lymphocyte activation as evidenced by decreased proliferation of T cells and cytokine secretion by these cells in the presence of B7-H3. The invention is yet further based, in part, on the discovery of specific regions within the B7-H3 genes that are currently undergoing purifying evolutionary selection.

In one aspect, the present disclosure provides in vitro, in vivo, and ex vivo methods of modulating immune responses, including methods of treating humans or animals. In some embodiments, such methods comprise a step of contacting a lymphocyte, such as a T cell, with a B7-H3 agent, wherein the B7-H3 agent may be (a) a derivative of B7-H3 such as a soluble form of B7-H3; (b) an antibody against B7-H3; (c) an antibody against a B7-H3 receptor; or (d) a nucleic acid comprising at least a portion of the B7-H3 mRNA or a complement thereto. In certain embodiments, the B7-H3 agent is coupled with the primary (stimulatory, antigen-specific) signal.

In particular embodiments, the methods of the invention are used to treat or prevent immune disorders susceptible to treatment with such compositions. Specifically, such disorders include but are not limited to immunologic disorders, including autoimmune disorders (e.g., rheumatoid arthritis (RA), psoriasis, multiple sclerosis (MS), inflammatory bowel disease (IBD), Crohn's disease, systemic lupus erythematosis (SLE), type I diabetes), transplant rejection, graft-versus-host disease (GVHD), hyperproliferative immune disorders, cancers, immunosuppressive disorders, various infectious diseases, etc. Thus, in certain embodiments, the methods of the invention comprise identifying a subject in need of inhibiting lymphocyte activation, and administering a B7-H3 agent that is an agonist to the subject. In other embodiments, the methods comprise identifying a subject in need of enhancing lymphocyte activation, and administering a B7-H3 agent that is an antagonist to the subject.

Antibodies used in the methods of the invention fall into two groups: (1) antibodies against B7-H3 and (2) antibodies against a B7-H3 receptor. These antibodies may: (a) specifically bind to B7-H3 thereby blocking the interaction of B7-H3 with its receptor; (b) specifically bind to a B7-H3 receptor thereby blocking its interaction with B7-H3; or (c) perform both (a) and (b). Depending on the desired effect, the antibodies may be used in alternative configurations to either enhance or inhibit immune responses. In some embodiments, antibodies are administered to antagonize the biological activity of naturally expressed B7-H3.

The disclosure further provides methods that involve compositions comprising soluble forms of B7-H3. In some embodiments, a soluble form of B7-H3 comprises less than full length B7-H3 and does not include the transmembrane and the intracellular domains of B7-H3. In further embodiments, a soluble form of B7-H3 comprises at least one V domain of B7-H3, and optionally at least one C domain of B7-H3. A soluble form may comprise at least 2, 3, 4, or 5 V domains, and optionally at least 1, 2, 3, 4, or 5 C domains. In yet further embodiments, a soluble form of B7-H3 may comprise: (a) a first amino acid sequence derived from the extracellular domain of B7-H3 and (b) a second amino acid sequence derived from the constant region of an antibody. The first amino acid sequence is derived from all or a portion of the B7-H3 extracellular domain and (a) competitively inhibits binding of a naturally occurring form of B7-H3 to its receptor and/or (b) has a negative costimulatory activity. In some embodiments, the first amino acid sequence comprises a sequence as set out in SEQ ID NO:15. In certain embodiments, the first amino acid sequence is identical to or is substantially identical to amino acids 23-244 of SEQ ID NO:14, or amino acids 23-462 of SEQ ID NO:12. In an illustrative embodiment, the soluble form of B7-H3 comprises a sequence as in SEQ ID NO:12 or SEQ ID NO:14.

The disclosure also provides methods involving therapeutic and nontherapeutic uses of nucleic acids or polypeptides encoded by such nucleic acids, where the nucleotide sequence of such nucleic acid is selected from: (a) a nucleotide sequence of SEQ ID NO:1, SEQ ID NO:3, SEQ ID NO:5, or a portion thereof; and (b) a nucleic acid that is at least 60, 80, 100, 120, or 140 nucleotides long and hybridizes to the nucleic acid of (a) under defined conditions, wherein the nucleic acid encodes an expression product having a negative costimulatory activity. In certain embodiments, such a nucleic acid encodes an amino acid sequence as in SEQ ID NO:15. In an illustrative embodiment, the nucleic acid comprises a sequence substantially as in SEQ ID NO:11 or SEQ ID NO:13.

The methods of the invention also encompass the use of short interfering RNAs and antisense nucleic acids to reduce the expression of B7-H3 in order to enhance immune response.

The invention also encompasses vectors that contain any of the foregoing nucleic acids and host cells containing any such vector.

It is to be understood that both the foregoing general description and the following detailed description are exemplary and explanatory only and are not restrictive of the invention, as claimed.

### BRIEF DESCRIPTION OF THE FIGURES

FIG. 1A depicts human and mouse B7-H3 sequence comparisons. Sequence alignment of deduced translated human B7-H3 VC, human B7-H3 VCVC, and mouse B7-H3 gene products. Dark bars above sequence alignment denote exon domains demarcated by genomic sequences. Arrows below peptide sequence denote corresponding nucleic acid positions used in oligonucleotide primers used for cross-species amplification.

FIG. 1B depicts genomic organization of human and mouse B7-H3 gene loci. Assemblies are based on selected portions of Celera human genomic axis GA_x2HTBL4SSTP and Celera mouse genomic axis GA_x5J8B7W7NM9. Bars denote relative locations of Alu and SVA complex repeats, simple repeats, transcript exon structure, and domain name.

FIG. 2A shows results of B7-H3 RT-PCR on human samples. Three separate panels of first strand human cDNA were amplified with PCR primers common to both B7-H3 VC and B7-H3 VCVC sequences and detected with oligonucleotides to V₁ domain. Expected sizes of amplified products are as indicated.

FIG. 2B shows results of B7-H3 RT-PCR on mouse samples. A mouse panel consisting of adult and embryonic cDNA was analyzed for the presence of B7-H3 transcripts. Predominant specific hybridization of a ˜1 kb band is consistent with the presence of the single VC form of mouse B7-H3.

FIG. 3A-3B demonstrate a costimulatory effect of B7 as measured by proliferation of activated T cells. B7-H3 activation of T cells results in attenuation of proliferation and cytokine production. FIG. 3A depicts results of proliferation assays for CHO.HLA-DR2 cells expressing GFP, B7-1, B7-2, whereas FIG. 3B shows results of proliferation assays for CHO.HLA-DR2 cells expressing GFP, B7-H3 VCVC, or B7-H3 VC. CHO.HLA-DR2 transfectants (1.25×10⁴ cells/well) were incubated with CD4⁺ T cells (10⁵ cells/well) in the presence of soluble anti-CD3 antibody (1 μg/ml) and titrated concentrations of anti-CD28 antibody. Proliferation was measured at 72 hours. Responses for CD4⁺ T cells plus CHO.HLA-DR2 transfectants in the absence of anti-CD3 antibody were below 300 CPM.

FIG. 4 demonstrates inhibitory effect of B7-H3 on cytokine production by activated T cells. CHO.HLA-DR2 cells expressing GFP, B7-H3 VCVC, or B7-H3 VC (1.25×10⁴ cells/well) were incubated with CD4⁺ T cells (10⁵ cells/well) in the presence of soluble anti-CD3 antibody (1 μg/ml) and titrated concentrations of anti-CD28 antibody (0.5 ng/ml). Supernatants were harvested from T cells cultures stimulated with either CHO.HLA-DR2 GFP, B7-H3 VCVC, or B7-H3 VC in the presence of anti-CD3 (1 μg/ml) and anti-CD28 (0.5 ng/ml) antibodies. Cytokine production was measured at 72 hours using multiplex ELISA screening.

FIG. 5 demonstrates that B7-H3 VC and B7-H3 VCVC deliver a negative signal to human CD4⁺ T cells as measured by inhibition of cell proliferation. Purified CD4⁺ cells (10⁵ cells/well) were activated with anti-CD3 antibody (1 μg/10⁷ microspheres) and B7-H3-Ig (VCVC or VC; 4 μg/10⁷ microspheres) on CIS or TRANS microspheres. CIS microspheres coated with both anti-CD3 antibody (1 μg/10⁷ microspheres) and B7-H3-Ig (VCVC or VC) at 4 μg/10⁷ microspheres. TRANS microspheres consisted of a mix of two types of microspheres: (a) microspheres coated with anti-CD3 antibody (1 μg/10⁷ microspheres) and (b) microspheres coated with B7-H3 (VCVC or VC). To maintain equal microsphere-to-cell ratio, microspheres coated control murine Ig were added to achieve a total protein concentration of 5 μg/10⁷ beads. Proliferation was measured at 72 hours.

FIGS. 6A-6C demonstrate that B7-H3 VC and B7-H3 VCVC deliver a negative signal to human CD4⁺ T cells as measured by inhibition of cytokine secretion. Purified CD4⁺ cells (10⁵ cells/well) were activated with anti-CD3 antibody (1 μg/10⁷ microspheres) and B7-H3-Ig (VCVC or VC; 4 μg/10⁷ microspheres) on CIS or TRANS microspheres. CIS microspheres coated with both anti-CD3 (1 μg/10⁷ microspheres) and B7-H3-Ig (VCVC or VC) at 4 μg/10⁷ microspheres. TRANS microspheres consisted of a mix of two types of microspheres: (a) microspheres coated with anti-CD3 antibody (1 μg/10⁷ microspheres) and (b) microspheres coated with B7-H3 (VCVC or VC). To maintain equal microsphere-to-cell ratio, microspheres coated control murine Ig were added to achieve a total protein concentration of 5 μg/10⁷ beads. The amount of cytokines in the supernatants was measured at 72 hours using multiplex ELISA screening: TNF-α (FIG. 6A), IFN-γ (FIG. 6B), and GM-CSF (FIG. 6C).

### BRIEF DESCRIPTION OF THE SEQUENCES

SEQ ID NO:1 and SEQ ID NO:2 represent, respectively, nucleic acid and amino acid full-length sequences of human B7-H3 VC.

SEQ ID NO:3 and SEQ ID NO:4 represent, respectively, nucleic acid and amino acid full-length sequences of mouse B7-H3.

SEQ ID NO:5 and SEQ ID NO:6 represent, respectively, nucleic acid and amino acid full-length sequences of human B7-H3 VCVC.

SEQ ID NO:7 represents an amino acid sequence of human B7-H3 (amino acid 28-139 of B7-H3 VC or B7-H3 VCVC).

SEQ ID NO:8 represents amino acids conserved between the V1 and V2 regions of human B7-H3 VCVC, i.e., between amino acids 28-139 and 246-357 of SEQ ID NO:6.

SEQ ID NO:9 and SEQ ID NO:10 represent respectively nucleic acid and amino acid sequences of a fusion polypeptide containing the oncostatin M signal sequence (amino acids 1-22 of SEQ ID NO:10), the extracellular domain of human B7-H3 VC (amino acids 23-244 of SEQ ID NO:10), and the constant region of mouse IgG₂ₐₘ (amino acids 245-482 of SEQ ID NO:10).

SEQ ID NO:11 and SEQ ID NO:12 represent respectively nucleic acid and amino acid sequences of a fusion polypeptide containing the oncostatin M signal sequence (amino acids 1-22 of SEQ ID NO:12), the extracellular domain of human B7-H3 VCVC (amino acids 23-462 of SEQ ID NO:12), and the constant region of mouse IgG₂ₐₘ (amino acids 463-700 of SEQ ID NO:12).

SEQ ID NO:13 and SEQ ID NO:14 represent, respectively, nucleic acid and amino acid sequences of a fusion polypeptide containing the oncostatin M signal sequence (amino acids 1-22 of SEQ ID NO:14), the extracellular domain of mouse B7-H3 VC (amino acids 23-244 of SEQ ID NO:14), and the constant region of mouse IgG₂ₐₘ (amino acids 245-482 of SEQ ID NO:14).

SEQ ID NO:15 represents conserved amino acids in the Ig V-like domain(s) of mammalian B7-H3.

SEQ ID NOs:16-22 represent individual highly conserved regions in the Ig V-like domain(s) of mammalian B7-H3.

SEQ ID NOs:23-35 represent PCR primers employed for isolation of B7-H3 sequences as described in the Examples.

### DETAILED DESCRIPTION OF THE INVENTION

1. Definitions

In order for the present invention to be more readily understood, certain terms are defined herein. Additional definitions are set forth throughout the detailed description.

The term “antibody,” as used herein, refers to an immunoglobulin or a part thereof, and encompasses any polypeptide comprising an antigen-binding site regardless of the source, method of production, and other characteristics. The term includes but is not limited to polyclonal, monoclonal, monospecific, polyspecific, non-specific, humanized, single-chain, chimeric, synthetic, recombinant, hybrid, mutated, and CDR-grafted antibodies. The term “antigen-binding domain” refers to the part of an antibody molecule that comprises the area specifically binding to or complementary to a part or all of an antigen. Where an antigen is large, an antibody may only bind to a particular part of the antigen. The “epitope,” or “antigenic determinant” is a portion of an antigen molecule that is responsible for specific interactions with the antigen-binding domain of an antibody. An antigen-binding domain may be provided by one or more antibody variable domains (e.g., a so-called Fd antibody fragment consisting of a V_{H} domain). An antigen-binding domain comprises an antibody light chain variable region (V_{L}) and an antibody heavy chain variable region (V_{H}).

The term “anti-B7-H3 antibody,” or “antibody against B7-H3,” refers to any antibody that specifically binds to at least one epitope of at least one B7-H3 form, including but not limited to B7-H3 VC and B7-H3 VCVC. The terms “anti-B7-H3 receptor antibody” and “antibody against a B7-H3 receptor” refer to any antibody that specifically binds to at least one epitope of a receptor for B7-H3.

As used herein, the term “B7-H3,” unless otherwise stated, refers to any and all forms of B7-H3, including but not limited to VC and VCVC. The term “B7-H3 agent” refers to any compound capable of modulating biological activity of B7-H3. The term “modulating” and its cognates refer to a reduction or an increase in biological activity of B7-H3, e.g., the activity associated with the effect exerted by naturally expressed B7-H3 on a lymphocyte expressing a B7-H3 receptor. A reduction or an increase in biological activity is preferably at least 10%, 20%, 30%, 40%, 50%, 60%, 70%, 80%, 90%, or more. A B7-H3 agent that causes such a reduction is referred to as “antagonist,” while a B7-H3 agent that causes such an increase is referred to as “agonist.” It will be understood that an antagonist of B7-H3 would counteract the “negative costimulatory signal” exerted by naturally expressed B7-H3 on a lymphocyte expressing a B7-H3 receptor, whereas an agonist would enhance such a “negative costimulatory signal.” Therefore, an antagonist of B7-H3 generally causes an increase in lymphocyte activation (e.g., as measured by cell proliferation and/or cytokine secretion), whereas an agonist of B7-H3 generally cause a decrease in the same.

The term “biological activity” refers to a function or set of functions (or the effect to which the function is attributed to) performed by a molecule in a biological system, which may be in vivo or in vitro. Biological activity may be assessed by, for example, the effect on lymphocyte proliferation, survival, and function (e.g., cytokine secretion), cluster of differentiation marker expression, gene expression at the transcriptional, translational, or post-translational levels, or the effect on autoantibody production, etc.

The term “costimulation” and its cognates refer to signaling events between receptor/ligand pairs of cell surface molecules on the responder lymphocyte and an “accessory” cell (e.g., an antigen presenting cell (APC) in the case of T cell activation, or a helper T cell for B cell activation) to allow lymphocyte activation. The terms “negative costimulation,” “negative costimulatory signal,” “inhibitory signal,” “negative costimulatory activity,” and their cognates refer to signaling events that inhibit lymphocyte activation relative to that in the absence of such signals. It will be understood that an activated T cell may be a helper cell (i.e., CD4⁺), a cytotoxic, or a suppressor cell (i.e., CD8⁺). Negative costimulatory activity can be measured using standard techniques and, without limitation, as described in the Examples. In particular, the presently disclosed B7-H3 agents inhibit lymphocyte activation, which can be measured by (a) cell proliferation and/or (b) cytokine secretion.

The term “derivative,” “derived from,” and their cognates, when used in reference to an amino acid or a nucleotide sequence, refers to a sequence that is identical or substantially identical to all or a portion of a parent sequence and can be actually obtained from the parent sequence, for example, by way of amino acid or nucleotide substitution, deletion, or addition, or other modifications.

The term “hybridization under defined conditions” refers to conditions for hybridization and washes under which nucleotide sequences that are significantly identical or homologous to each other remain bound to each other. The conditions are such that sequences, which are at least 50, 100, 150, 300, or more nucleotides long and at least 70%, more preferably at least 80%, even more preferably at least 85-90% identical, remain bound to each other. The percent identity can be determined as described in Altschul et al. (1997) Nucleic Acids Res., 25:3389-3402. Nonlimiting examples of low, moderate, and high stringency hybridization conditions are provided in subsequent sections.

The term “immunologic disorder” refers to disorders and conditions in which an immune response is aberrant. The aberrant response can be due to (a) abnormal proliferation, maturation, survival, differentiation, or function of immune cells such as, for example, T or B cells. Such disorders include but are not limited to autoimmune disorders (e.g., rheumatoid arthritis (RA), psoriasis, multiple sclerosis (MS), inflammatory bowel disease (IBD), Crohn's disease, systemic lupus erythematosis (SLE), type I diabetes), transplant rejection, graft-versus-host disease (GVHD), hyperproliferative immune disorders, and immunosuppressive disorders. In particular, the disclosure provides methods that involve compositions comprising B7-H3 agents such as soluble forms of B7-H3 or antibodies against B7-H3 or against its receptor.

The term “isolated” refers to a molecule that is substantially free of its natural environment. For instance, an isolated protein is substantially free of cellular material or other proteins from the cell or tissue source from which it is derived. The term “isolated” also refers to preparations where the isolated protein is sufficiently pure to be administered as a pharmaceutical composition, or at least 70-80% (w/w) pure, more preferably, at least 80-90% (w/w) pure, even more preferably, 90-95% pure; and, most preferably, at least 95%, 96%, 97%, 98%, 99%, or 100% (w/w) pure. The term “isolated,” as used herein, also refers to preparations that are substantially endotoxin-free, i.e., the endotoxin levels are below 500, 300, 200, 100, 50, 10, 5, 1, 0.5, 0.1, 0.05, 0.01 EU/ml, or below a detectable level.

The term “mammal” refers to any animal classified as such, including humans.

The term “primary stimulatory signal” refers to a stimulatory signal delivered to a lymphocyte that confers specificity to the immune response and is mediated by the antigen-specific receptor (TcR—on T cells, and BcR—on cells) upon recognition of the antigenic peptide-MHC complex.

The terms “treatment,” “therapeutic method,” and their cognates refer to both therapeutic treatment and prophylactic/preventative measures. Those in need of treatment may include individuals already having a particular medical disorder as well as those at risk for the disorder (i.e., those who are likely to ultimately acquire the disorder). A therapeutic method results in prevention or amelioration of symptoms or an otherwise desired biological outcome and may be evaluated by improved clinical signs (e.g., PASI as described in the Examples), delayed onset of disease, reduced/elevated levels of lymphocytes and/or antibodies, etc.

The terms “therapeutic compound” and “therapeutic,” as used herein, refer to any compound capable of ameliorating clinical manifestations of a disorder, or to produce a desired biological outcome.

The terms “therapeutically effective dose” and “therapeutically effective amount” refer to that amount of a compound that results in prevention or amelioration of symptoms in a patient or a desired biological outcome, e.g., improved clinical signs (e.g., PASI as described in the Examples), delayed onset of disease, reduced/elevated levels of lymphocytes and/or antibodies, etc. The effective amount can be determined as described in the subsequent sections.

The term “specifically binding” and its cognates mean that two molecules form a complex that is relatively stable under physiologic conditions. Specific binding is characterized by a high affinity and a low to moderate capacity. Nonspecific binding usually has a low affinity with a moderate to high capacity. Typically, the binding is considered specific when the affinity constant Kₐ is higher than 10⁶ M⁻¹, or preferably higher than 10⁸ M⁻¹. If necessary, nonspecific binding can be reduced without substantially affecting specific binding by varying the binding conditions. Such conditions are known in the art, and a skilled artisan using routine techniques can select appropriate conditions. The conditions are usually defined in terms of protein concentration, ionic strength of the solution, temperature, time allowed for binding, concentration of unrelated molecules (e.g., serum albumin, milk casein), etc.

The phrase “substantially identical” means that a relevant amino acid sequence is at least 70%, 75%, 80%, 85%, 90%, 95%, 97%, 98%, 99%, or 100% identical to a given sequence. By way of example, such sequences may be variants derived from various species, or they may be derived from the given sequence by truncation, deletion, amino acid substitution or addition. Percent identity between two amino acid sequences is determined by standard alignment algorithms such as, for example, Basic Local Alignment Tool (BLAST) described in Altschul et al. (1990) J. Mol. Biol., 215:403-410, the algorithm of Needleman et al. (1970) J. Mol. Biol., 48:444-453; the algorithm of Meyers et al. (1988) Comput. Appl. Biosci., 4:11-17; or Tatusova et al. (1999) FEMS Microbiol. Lett., 174:247-250, etc. Such algorithms are incorporated into the BLASTN, BLASTP and “BLAST 2 Sequences” programs (see www.ncbi.nlm.nih.gov/BLAST). When utilizing such programs, the default parameters can be used. For example, for nucleotide sequences the following settings can be used for “BLAST 2 Sequences”: program BLASTN, reward for match 2, penalty for mismatch -2, open gap and extension gap penalties 5 and 2 respectively, gap x_dropoff 50, expect 10, word size 11, filter ON. For amino acid sequences the following settings can be used for “BLAST 2 Sequences”: program BLASTP, matrix BLOSUM62, open gap and extension gap penalties 11 and 1 respectively, gap x_dropoff 50, expect 10, word size 3, filter ON.

The terms “polynucleotide,” “oligonucleotide,” and “nucleic acid” refer to deoxyribonucleic acid (DNA) and, where appropriate, to ribonucleic acid (RNA), or peptide nucleic acid (PNA). The term should also be understood to include nucleotide analogs, and single or double stranded polynucleotides (e.g., siRNA). Examples of polynucleotides include but are not limited to plasmid DNA or fragments thereof, viral DNA or RNA, antisense RNA, etc. The term “plasmid DNA” refers to double stranded DNA that is circular. “Antisense,” as used herein, refers to a nucleic acid capable of hybridizing to a portion of a coding and/or noncoding region of mRNA by virtue of sequence complementarity, thereby interfering with translation from the mRNA. The terms “siRNA” and “RNAi” refer to a nucleic acid which is a double stranded RNA that has the ability to induce degradation of mRNA thereby “silencing” gene expression. Typically, siRNA is at least 15-50 nucleotides long, e.g., 20, 21, 22, 23, 24, 25, 26, 27, 28, 29, or 30 nucleotides in length.

The term “V domain” (singular or plural), unless specifically stated, refers to the first Ig-like variable domain (V₁) and/or the second Ig-like domain (V₂) in the protein or genomic sequence of B7-H3, regardless of the species of origin (e.g., any sequence comprising SEQ ID NO:15 and a nucleotide sequence encoding it; or a sequence substantially identical to SEQ ID NO:7 and a nucleotide sequence encoding it). Likewise, the term “C domain” (singular or plural), unless specifically stated, refers to the first Ig-like constant domain (C₁) and/or the second Ig-like constant domain (C₂) in the protein or genomic sequence of B7-H3, regardless of the species of origin. Unless context requires otherwise, references to V and C domains should be understood to encompass the protein domains, nucleotide sequences encoding therefor, and pseudo-exon sequences corresponding to the coding sequences (e.g., C_{ψ} and V_{ψ} of rodent genomic sequences).

2. B7-H3 Agents

In one aspect, the present invention relates to the use of B7-H3 agents in modulation of immune responses. The present invention is based, in part, on the discovery and demonstration that the VCVC form of B7-H3 accounts for the majority of B7-H3 transcripts seen across multiple tissues while the VC form of B7-H3 is only a minor transcript. The invention is further based, in part, on the discovery and demonstration that both forms of B7-H3, VC and VCVC, exhibit an inhibitory effect on T cell activation as evidenced by decreased proliferation and cytokine secretion by the cells in the presence of B7-H3. The invention is yet further based, in part, on the discovery of specific regions within the B7-H3 genes that are currently undergoing purifying evolutionary selection.

Portions of mouse, human, monkey, and hamster genomic V-exons were aligned using ClustalW of the Align module of Vector NTI version 8.0. Regions that exhibited 100% sequence identity in alignment greater or equal to nine nucleotides were chosen as the most highly conserved nucleotide positions. These eleven conserved regions are represented in SEQ ID NO:15.

In certain embodiments, compositions used in the methods of the invention comprise a B7-H3 agent that antagonizes or agonizes the biological activity of naturally occurring B7-H3. In some embodiments, the B7-H3 agent is proteinaceous, i.e., it comprises amino acids linked by peptide bonds. Proteinaceous B7-H3 agents include but are not limited to soluble forms of B7-H3, including B7-H3-Ig fusions, antibodies against B7-H3, and antibodies against a B7-H3 receptor. In other embodiments, compositions used in the methods of the invention comprise nonproteinaceous B7-H3 agents, such as nucleic acids, small molecule inhibitors, etc. In particular, the presently disclosed B7-H3 agents modulate lymphocyte activation as measured by one or more of the following: (a) lymphocyte proliferation; and (b) cytokine secretion (e.g., interleukin (IL)-10, tumor necrosis factor (TNF)-α, interferon (IFN)-γ, and granulocyte-macrophage-colony stimulating factor (GM-CSF)). In some embodiments, B7-H3 agents possess pharmacokinetic properties that make it suitable for therapeutic use, e.g., sufficiently long circulatory half-life and/or acceptable protection from proteolytic degradation.

2.1 Antibodies

Antibodies used in the methods of the invention fall into two groups: (1) antibodies against B7-H3 and (2) antibodies against a B7-H3 receptor. In various embodiments, antibodies used in the methods of the invention specifically bind to at least one of: (a) B7-H3; (b) B7-H3 receptor; (c) V domain in B7-H3; (d) C domain in B7-H3; and (e) polypeptide comprising SEQ ID NO:15, SEQ ID NO:2, SEQ ID NO:4, SEQ ID NO:6, or SEQ ID NO:7. Such antibodies may (a) specifically bind to B7-H3 thereby blocking the interaction of B7-H3 with its receptor; (b) specifically bind to a B7-H3 receptor thereby blocking its interaction with B7-H3; or (c) perform both (a) and (b). Depending on the desired effect, the antibodies may be used in alternative configurations to either enhance (as an antagonist of B7-H3 biological activity) or inhibit immune responses (as an agonist of B7-H3 biological activity) as described in subsequent sections.

Antibodies can be made, for example, by traditional hybridoma techniques (Kohler and Milstein (1975) Nature, 256:495-499), recombinant DNA methods (U.S. Pat. No. 4,816,567), or phage display techniques using antibody libraries (Clackson et al. (1991) Nature, 352:624-628; Marks et al. (1991) J. Mol. Biol., 222:581-597). For various other antibody production techniques, see, e.g., Antibodies: A Laboratory Manual, eds. Harlow et al., Cold Spring Harbor Laboratory, 1988; and Antibody Engineering, 2nd ed., Oxford University Press, ed. Borrebaeck, 1995. For administration to humans, antibodies may be fully human or humanized. In certain embodiments, antibodies may have an altered or mutated Fc region as described in subsequent sections.

2.2 Soluble Forms of B7-H3

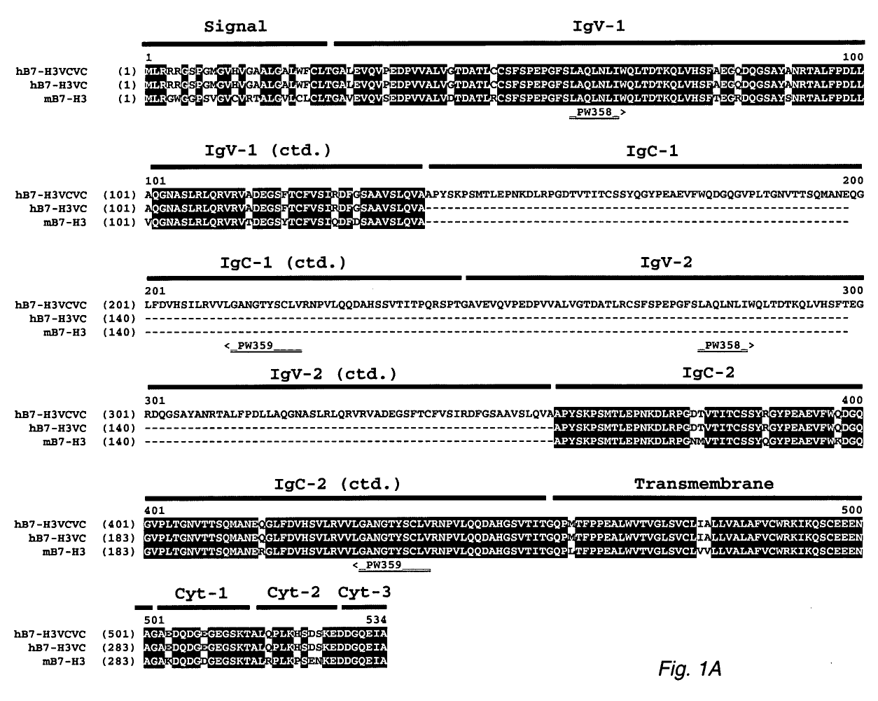

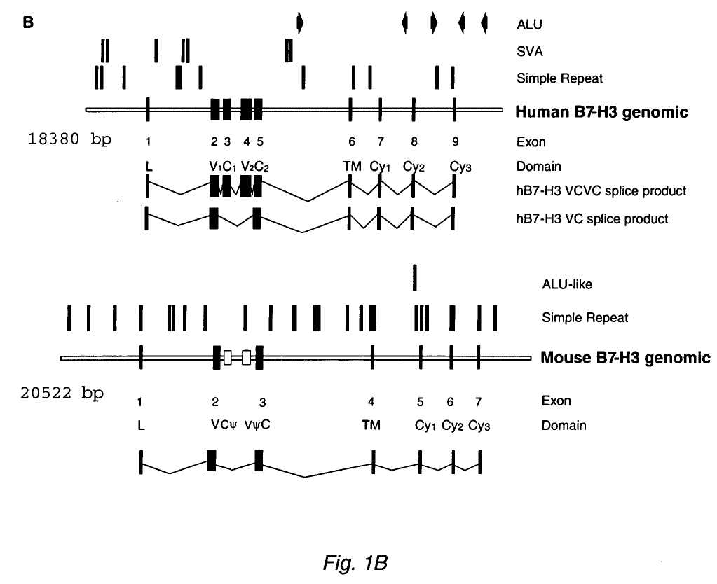

The methods of the invention involve a use of soluble forms of B7-H3 that inhibit lymphocyte activation. In some embodiments, a soluble form of B7-H3 comprises less than full length B7-H3 and does not include the transmembrane and the intracellular domains of B7-H3. Such a soluble form may also not include a signal sequence. For illustration only, and not to be limiting, these domains can be delineated in human and mouse B7-H3 as depicted in FIG. 1A.

In certain embodiments, a soluble form comprises an amino acid sequence as in SEQ ID NO:15 or SEQ ID NO:7. In yet further embodiments, a soluble form of B7-H3 comprises at least one V domain of B7-H3, and optionally at least one C domain of B7-H3. A soluble form may comprise at least 2, 3, 4, or 5 V domains, and optionally at least 1, 2, 3, 4, or 5 C domains.

In further embodiments, a soluble form of B7-H3 may comprise (a) a first amino acid sequence derived from the extracellular domain of B7-H3 and (b) a second amino acid sequence derived from the constant region of an antibody. The first amino acid sequence is derived from all or a portion of the B7-H3 extracellular domain and (a) competitively inhibits binding of a naturally occurring form of B7-H3 to its receptor and/or (b) has a negative costimulatory activity.

In some embodiments, the first amino acid sequence comprises a sequence as set out in SEQ ID NO:15. In certain embodiments, the first amino acid sequence is identical to or is substantially identical to amino acids 23-244 of SEQ ID NO:14, or amino acids 23-462 of SEQ ID NO:12. In an illustrative embodiment, the soluble form of B7-H3 comprises a sequence as in SEQ ID NO:12 or SEQ ID NO:14.

The second amino acid sequence may be derived from the constant region of an antibody, such as the Fc portion. In some embodiments, the second amino acid sequence is derived from the Fc portion of an IgG. In related embodiments, the Fc portion is derived from IgG that is IgG₁, IgG₄, or another IgG isotype. In nonlimiting illustrative embodiments, the second sequence is derived from mouse IgG₂ₐₘ.

In certain embodiments, the second amino acid sequence is linked to the C-terminus or the N-terminus of the first amino acid sequence, with or without being linked by a linker sequence. The exact length and sequence of the linker and its orientation relative to the linked sequences may vary. The linker may, for example, comprise one or more Gly-Ser. The linker may be at least 2, at least 10, as least 20, at least 30, amino acids long and is selected based on properties desired such as solubility, length, steric separation, immogenicity, etc.

2.3 Derivatives of Proteinaceous B7-H3 Agents

Derivatives of proteinaceous B7-H3 agents (including soluble forms of B7-H3, antibodies against B7-H3, and antibodies against B7-H3 receptor) can be made by altering their amino acids sequences by substitutions, additions, and/or deletions/truncations or by introducing chemical modification that result in functionally equivalent or molecules. It will be understood by one of ordinary skill in the art that certain amino acids in a sequence of any protein may be substituted for other amino acids without adversely affecting the activity of the protein.

Various changes may be made in the amino acid sequences of the proteinaceous B7-H3 agents of the invention or DNA sequences encoding therefor without appreciable loss of their biological activity, function, or utility. The use of such derivatives is within the scope of the present invention. In a specific embodiment, the derivative is functionally active, i.e., capable of exhibiting one or more activities associated with the extracellular domain of the naturally occurring B7-H3, e.g., as set out in SEQ ID NO:2, SEQ ID NO:4, or SEQ ID NO:6. Substitutes for an amino acid within the sequence may be selected from other members of the class to which the amino acid belongs (see Table 1). Furthermore, various amino acids are commonly substituted with neutral amino acids, e.g., alanine, leucine, isoleucine, valine, proline, phenylalanine, tryptophan, and methionine (see, e.g., MacLennan et al. (1998) Acta Physiol. Scand. Suppl. 643:55-67; Sasaki et al. (1998) Adv. Biophys. 35:1-24).

TABLE 1

| Original | Exemplary | Typical |
|---|---|---|
| Residues | Substitutions | Substitutions |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, 1,4-Diamino-butyric Acid, Gln, Asn | Arg |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, Norleucine | Leu |

B7-H3 agents may be chemically coupled, or conjugated, to other proteins and pharmaceutical agents. Such modifications may be designed to alter the pharmacokinetics and/or biodistribution of the resultant composition. The B7-H3-Ig and antibodies of the invention may also be glycosylated, pegylated, or linked to another nonproteinaceous polymer, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337. The B7-H3-Ig and antibodies may be chemically modified by covalent conjugation to a polymer to increase their circulating half-life, for example. Exemplary polymers, and methods to attach them to peptides, are also shown in U.S. Pat. Nos. 4,766,106; 4,179,337; 4,495,285; and 4,609,546.

B7-H3 agents that comprise the Fc portion of an antibody, such as B7-H3-Ig fusions, or antibodies of used in the methods of the invention may further be modified in the Fc region to minimize the effector function. Such modifications include changing specific amino acid residues which alter binding to an Fc receptor (Lund et al. (1991) J. Immun., 147:2657-2662 and Morgan et al. (1995) Immunology, 86:319-324), or changing the species from which the constant region is derived. Antibodies and B7-H3-Ig fusions may have mutations in the C_{H}2 region of the heavy chain that reduce effector function, i.e., Fc receptor binding and complement activation. For example, antibodies and B7-H3-Ig fusions may have mutations such as those described in U.S. Pat. Nos. 5,624,821 and 5,648,260. In the IgG₁ or IgG₂ heavy chain, for example, such mutations may be made at amino acid residues corresponding to amino acids 234 and 237 in the full-length sequence of IgG₁ or IgG₂. Antibodies and B7-H3-Ig fusions may also have mutations that stabilize the disulfide bond between the two heavy chains of an immunoglobulin, such as mutations in the hinge region of IgG₄, as disclosed in Angal et al. (1993) Mol. Immunol., 30:105-108.

In certain embodiments, additional fusions of any of B7-H3-Ig of the invention to amino acid sequences derived from other proteins may be constructed for use in the methods of the invention. Desirable fusion sequences may be derived from proteins having biological activity different from that of B7-H3, for example, cytokines, growth and differentiation factors, enzymes, hormones, other receptor components, etc.

The B7-H3 agents (proteinaceous and nonproteinaceous) may also be tagged with a detectable or functional label. Detectable labels include radiolabels such as ¹³¹I or ⁹⁹Tc, which may be attached using conventional chemistry. Detectable labels further include enzyme labels, e.g., horseradish peroxidase or alkaline phosphatase and detectable moieties such as biotin or avidin.

Derivatives can be produced by various techniques well known in the art, including recombinant and synthetic methods (Maniatis (1990) Molecular Cloning, A Laboratory Manual, 2nd ed., Cold Spring Harbor Laboratory, Cold Spring Harbor, N.Y.; and Bodansky et al. (1995) The Practice of Peptide Synthesis, 2nd ed., Spring Verlag, Berlin, Germany).

2.4 Nucleic Acids

The disclosure also provides methods involving therapeutic and nontherapeutic uses of nucleic acids or polypeptides encoded by such nucleic acids, wherein the nucleotide sequence of such nucleic acid is chosen from (a) a nucleotide sequence of SEQ ID NO:1, SEQ ID NO:3, SEQ ID NO:5, or a portion thereof; and (b) a nucleic acid that is at least 60, 80, 100, 120, or 140 nucleotides long and hybridizes to the nucleic acid of (a) under defined conditions, wherein the nucleic acid encodes an expression product having a negative costimulatory activity.

In certain embodiments, such a nucleic acid encodes an amino acid sequence as in SEQ ID NO:15. In an illustrative embodiment, the nucleic acid comprises a sequence substantially as in SEQ ID NOs:11 or SEQ ID NO:13. In other embodiments, such a nucleic acid includes a nucleotide sequence that differs from SEQ ID NO:11 or SEQ ID NO:13 in that it has at least one synonymous substitution, i.e., the codon having the substitution encodes the same or a functionally equivalent amino acid residue as in SEQ ID NO:11 or SEQ ID NO:13.

In one embodiment, the defined conditions are low stringency conditions. In another embodiment, the defined conditions are moderate stringency conditions. In yet another embodiment, the defined conditions are high stringency conditions.

Appropriate hybridization conditions can be easily selected by those skilled in the art as exemplified in Ausubel et al. (1995), Current Protocols in Molecular Biology, John Wiley & Sons, sections 2, 4, and 6. Additionally, stringent conditions are described in Sambrook et al. (1989) Molecular Cloning: A Laboratory Manual, 2nd ed., Cold Spring Harbor Press, chapters 7, 9, and 11. A nonlimiting example of defined conditions of low stringency is as follows. Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10^{6 32}P-labeled probe is used. Filters are incubated in hybridization mixture for 18-20 h at 40° C. and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. Other conditions of low stringency well known in the art may be used (e.g., as employed for cross-species hybridizations).

A nonlimiting example of defined conditions of moderate stringency is as follows. Prehybridization of filters containing DNA is carried out for 7 h to overnight at 50° C. in buffer composed of 5×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 18-36 h at 50° C. in the prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 minutes. Other conditions of moderate stringency well known in the art may be used.

A nonlimiting example of defined conditions of high stringency is as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in the prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 minutes. Other conditions of high stringency well known in the art may be used.

B7-H3 agents may be obtained, isolated, and/or purified from their natural environment, in substantially pure or homogeneous form, or in the case of nucleic acid, free or substantially free of nucleic acid or genes origin other than the sequence encoding a polypeptide with the required function. Systems for cloning and expression of a polypeptide in a variety of different host cells are well known. Suitable host cells include bacteria, mammalian cells, and yeast and baculovirus systems. Mammalian cell lines available in the art for expression of a heterologous polypeptide include Chinese hamster ovary cells, HeLa cells, baby hamster kidney cells, NS0 mouse melanoma cells and many others. A common bacterial host is E. coli. For other cells suitable for producing, e.g., B7-H3-Ig, see Gene Expression Systems, eds. Fernandez et al., Academic Press, 1999.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids or viral, e.g., phage, or phagemid, as appropriate. For further details see, e.g., Molecular Cloning: A Laboratory Manual, Sambrook et al., 2nd ed., Cold Spring Harbor Laboratory Press, 1989. Many known techniques and protocols for manipulation of nucleic acid, for example, in preparation of nucleic acid constructs, mutagenesis, sequencing, introduction of DNA into cells and gene expression, and analysis of proteins, are described in detail in Current Protocols in Molecular Biology, eds. Ausubel et al., 2nd ed., John Wiley & Sons, 1992.

A nucleic acid can be fused to other sequences encoding additional polypeptide sequences, for example, sequences that function as a marker or reporter. Examples of marker or reporter genes include β-lactamase, chloramphenicol acetyltransferase (CAT), adenosine deaminase (ADA), aminoglycoside phosphotransferase (responsible for neomycin (G418) resistance), dihydrofolate reductase (DHFR), hygromycin-B-phosphotransferase (HPH), thymidine kinase (TK), lacZ (encoding β-galactosidase), xanthine guanine phosphoribosyltransferase (XGPRT), and many others known in the art.

The methods of the invention also encompass the use of short interfering RNAs (siRNA) and antisense oligonucleotides to reduce the expression of B7-H3 in order to enhance immune response. siRNA may be produced using standard techniques as described in Hannon (2002) Nature, 418:244-251; McManus et al. (2002) Nat. Reviews, 3:737-747; Heasman (2002) Dev. Biol., 243:209-214; Stein (2001) J. Clin. Invest., 108:641-644; and Zamore (2001) Nat. Struct. Biol., 8(9):746-750. Antisense nucleic acids may be produced using standard techniques as described in Antisense Drug Technology: Principles, Strategies, and Applications, 1st ed., ed. Crooke, Marcel Dekker, 2001.

3. Methods of Use

3.1 Methods of Modulating Immune Reponses

The disclosed B7-H3 agents can act as either agonists or antagonists of naturally expressed B7-H3, depending on the method of their use. The B7-H3 agents can be used to prevent, diagnose, or treat medical disorders in mammals (such as in humans). In vitro application of B7-H3 agents can be useful, for example, in production of activated lymphocytes for use in either studies on immune cell function or, for example, or for testing the biological activity of other B7-H3 agents. Such methods are detailed in the Examples.

In one aspect, the present invention relates to the use of B7-H3 and agonists and antagonists thereof in modulation of immune responses. The methods of the invention involve contacting a lymphocyte, e.g., T or B cell, with a B7-H3 agent in order to modulate (i.e., costinulate or inhibit) lymphocyte activation. In particular, the presently disclosed B7-H3 agents modulate lymphocyte activation as measured by one or more of the following: (a) lymphocyte proliferation; (b) cytokine secretion (e.g., interleukin (IL)-10, tumor necrosis factor (TNF)-α, interferon (IFN)-γ, and granulocyte-macrophage-colony stimulating factor (GM-CSF)). The methods can be performed in vitro, in vivo, or ex vivo.

The contacting step can occur before, during, or after activation of the lymphocyte. T cell activation can be effected, for example, by exposing the T cell to an antibody that binds to the TcR or one of the polypeptides of the CD3 complex that is physically associated with the TCR (e.g., using an anti-CD3 antibody; U.S. Pat. Nos. 6,405,696 and 5,316,763). Alternatively, the T cell can be exposed to either an alloantigen (e.g., a MHC alloantigen) on, for example, an antigen presenting cell (APC) (e.g., a dendritic cell, a macrophage, a monocyte, or a B cell) or an antigenic peptide produced by processing of a protein antigen by any of the above APC and presented to the T cell by MHC molecules on the surface of the APC. The T cell can be a CD4⁺ T cell or a CD8⁺ T cell. The B7-H3 agent can be added to the solution containing the cells, or it can be expressed on the surface of an APC, e.g., an APC presenting an alloantigen or an antigen peptide bound to an MHC molecule.

Furthermore, B7-H3 agents can be used to treat a subject at risk of or susceptible to a disorder or having a disorder associated with aberrant B7-H3 expression or function. Thus, in certain embodiments, the methods of the invention comprise identifying a subject in need of inhibiting lymphocyte activation, and administering a B7-H3 agonist to the subject. In other embodiments, the methods comprise identifying a subject in need of enhancing lymphocyte activation, and administering a B7-H3 antagonist to the subject.

When diminished immune response is desirable, B7-H3 agents may be used as agonists of B7-H3 in order to enhance the B7-H3-associated attenuation of the immune response. For example, B7-H3 agents can be used in methods of the invention for induction of tolerance to a specific antigen (e.g., a therapeutic protein). In one embodiment, tolerance is induced against a specific antigen by co-administration of antigen and a B7-H3 agent. For example, patients that received Factor VIII or Factor IX frequently generate antibodies to this protein, therefore co-administration of a B7-H3 agonist (e.g., B7-H3-Ig and nucleic acids encoding B7-H3 or its functional fragments) in combination with recombinant Factor VIII or Factor IX is expected to result in the downregulation of immune responses to this clotting factor. Additionally, a reduction in the level of immune response may be desirable, for example, in certain types of allergy or allergic reactions, autoimmune diseases (e.g., rheumatoid arthritis, psoriasis, type I diabetes mellitus, multiple sclerosis, inflammatory bowel disease, Crohn's disease, and systemic lupus erythematosis), tissue, skin and organ transplant rejection, and graft-versus-host disease (GVHD).

In certain embodiments, to achieve an agonistic effect, co-presentation, or coupling, (i.e., physical proximity) between positive (i.e., mediated by an antigen receptor, e.g., TcR or BcR) and negative (i.e., B7-H3) signals may be necessary. This may be achieved by immobilizing a B7-H3 agent on a support matrix which also carries a primary stimulatory molecule (e.g., andi-CD3 antibody). In such cases, the preferred distance is less than or comparable to the size of a naturally occurring antigen-presenting cell, i.e., less than 100 μm; more preferably, less than 50 μm; and most preferably, less than 20 μm. Alternatively, a B7-H3 agent can be coupled with a primary stimulatory molecule, e.g., by cross-linking via antibodies.

In some embodiments, the positive (activating) and the negative (inhibiting) signals are provided by a ligand or antibodies immobilized on solid support matrix, or a carrier. In various embodiments, the solid support matrix may be composed of polymer such as activated agarose, dextran, cellulose, polyvinylidene fluoride (PVDF). Alternatively, the solid support matrix may be based on silica or plastic polymers, e.g., as nylon, dacron, polystyrene, polyacrylates, polyvinyls, teflons, etc.

The matrix can be implanted into the spleen of a patient. Alternatively, the matrix may be used for the ex vivo incubation of T cells obtained from a patient, which are then separated and implanted back into the patient. The matrix may also be made from a biodegradable material such polyglycolic acid, polyhydroxyalkanoate, collagen, or gelatin so that they can be injected into the patient's peritoneal cavity, and dissolve after some time following the injection. The carrier can be shaped to mimic a cell (e.g., bead or microsphere).

Under certain circumstances, it may be desirable to elicit or enhance a patient's immune response in order to treat an immune disorder or cancer. The disorders being treated or prevented by the disclosed methods include but are not limited to infections with microbes (e.g., bacteria), viruses (e.g., systemic viral infections such as influenza, skin diseases such as herpes or shingles, and HIV), or parasites; and cancer (e.g., melanoma and prostate cancers).

In such circumstances, B7-H3 agents may be used to inhibit or reduce the downregulatory activity associated with B7-H3. In particular, B7-H3 antagonists (e.g., anti-B7-H3 antibody, antibody against a B7-H3 receptor, siRNA, and antisense nucleic acids to B7-H3) can be used for stimulation of T cell activation. In various embodiments, antibodies against B7-H3 or against a B7-H3 receptor inhibit binding of B7-H3 to cells expressing such a receptor with an IC₅₀ of less than 10 nM, and more preferably less than 5 nM, and most preferably less than 1 nM. IC₅₀ can be measured using standard techniques known in the art.

The compositions of the present invention are administered in therapeutically effective amounts. Generally, a therapeutically effective amount may vary with the subject's age, condition, and sex, as well as the severity of the medical condition of the subject. A therapeutically effective amount of proteinaceous B7-H3 agents ranges from 0.001 to 30 mg/kg, preferably from 0.01 to 25 mg/kg, from 0.1 to 20 mg/kg, or from 1 to 10 mg/kg body weight. The dosage may be adjusted, as necessary, to suit observed effects of the treatment. The antibodies and soluble forms of B7-H3 may given as a bolus dose. Continuous infusion may also be used after the bolus dose. The appropriate dose and regimen is chosen based on clinical indications by a treating physician.

Immune cells (e.g., activated T cells) can also be isolated from a patient and incubated ex vivo with a B7-H3 agent. For example, peripheral blood mononuclear cells (PBMC) can be withdrawn from a subject or a suitable donor and exposed ex vivo to an activating stimulus (see above) and a B7-H3 agent (whether in soluble form or attached to a sold support). The PBMC containing activated T cells are then introduced into the same or a different subject. Alternatively, isolated cells can be transfected with a nucleic acid and such transfected cell may then reintroduced into the subject. While such cells would preferably be hemopoietic cells (e.g., bone marrow cells, macrophages, monocytes, dendritic cells, T cells, or B cells) they could also be of another cell type including, without limitation, fibroblasts, epithelial cells, endothelial cells, keratinocytes. The use of hemopoietic cells may be advantageous in that such cells would be expected to home to, among others, lymphoid tissue (e.g., lymph nodes or spleen). In addition, if APC are used, the APC expressing the exogenous B7-H3 can be the same APC that presents an alloantigen or antigenic peptide to the relevant T cell. The B7-H3 agents can be secreted by the APC or expressed on its surface. Prior to returning the recombinant APC to the subject, they can optionally be exposed to sources of antigens or antigenic peptides of interest, e.g., those of tumors, infectious microorganisms, or autoantigens.

In some embodiments, B7-H3 agents are used to treat or prevent immune disorders susceptible to treatment with compositions of the invention which include but are not limited to immunologic disorders (e.g., rheumatoid arthritis (RA), psoriasis, multiple sclerosis (MS), inflammatory bowel disease (IBD), Crohn's disease, systemic lupus erythematosis (SLE), type I diabetes, transplant rejection, graft-versus-host disease (GVHD), hyperproliferative immune disorders, etc.), cancers, immunosuppressive disorders, and various infectious diseases. In particular, the disclosure provides methods that involve compositions comprising B7-H3 derivatives such as soluble forms of B7-H3 or antibodies against B7-H3 or against its receptor.

3.2 Screening Methods

B7-H3 agents can also be used in screening methods to identify therapeutic agents. A compound to be tested can be, for example, an anti-B7-H3 antibody, an antibody against a B7-H3 receptor, or a small organic molecule. In such a screening assay, a first binding mixture is formed by combining B7-H3-Ig and a cell expressing a B7-H3 receptor (e.g., an activated T cell); and the amount of binding between the two in the first binding mixture (M₀) is measured. A second binding mixture is also formed by combining B7-H3-Ig, a cell expressing a B7-H3 receptor, and an agent to be tested, and the amount of binding in the second binding mixture (M₁) is measured.

The amounts of binding in the first and second binding mixtures are then compared, for example, by calculating the M₁/M₀ ratio. The tested compound is considered to be capable of modulating a B7-H3-associated downregulation of immune responses if a decrease in binding in the second binding mixture as compared to the first binding mixture is observed. The formulation and optimization of binding mixtures is within the level of skill in the art, such binding mixtures may also contain buffers and salts necessary to enhance or to optimize binding, and additional control assays may be included in the screening assay of the invention. Compounds found to reduce the B7-H3 binding by at least 10% (i.e., M₁/M₀<0.9), preferably greater than 30% may thus be identified and then, if desired, secondarily screened for the capacity to ameliorate a disorder in other assays or animal models as described below. The strength of the binding can be measured using, for example, an enzyme-linked immunoadsorption assay (ELISA), radio-immunoassay (RIA), surface plasmon resonance-based technology (e.g., Biacore), all of which are techniques well known in the art.

The tested compound may then be further tested in vitro as described in the Examples or in an animal model (see, generally, Immunologic Defects in Laboratory Animals, eds. Gershwin et al., Plenum Press, 1981), for example, such as the following: the SWR X NZB (SNF1) mouse model (Uner et al. (1998) J. Autoimmune. Dis., 11 (3): 233-240), the KRN mouse (K/BxN) model (Ji et al. (1999) Immunol. Rev., 169: 139); NZB X NZW (B/W) mice, a model for SLE (Riemekasten et al. (2001) Arthritis Rheum., 44(10): 2435-2445); experimental autoimmune encephalitis (EAE) in mouse, a model for multiple sclerosis (Tuohy et al. (1988) J. Immunol., 141:1126-1130, Sobel et al. (1984) J. Immunol. 132:2393-2401, and Traugott (1989) Cell Immunol., 119:114-129); the NOD mouse model of diabetes (Baxter et al. (1991) Autoimmunity, 9(1):61-67), etc.).

Preliminary doses as, for example, determined according to animal tests, and the scaling of dosages for human administration is performed according to art-accepted practices. Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture assays or animal models. Levels in plasma may be measured, for example, by high performance liquid chromatography or ELISA. The effects of any particular dosage can be monitored by a suitable bioassay. Examples of dosages are: 0.1×IC₅₀, 0.5×IC₅₀, 1×IC₅₀, 5×IC₅₀, 10×IC₅₀, 50×IC₅₀, and 100×IC₅₀.

The data obtained from the cell culture assays or animal studies can be used in formulating a range of dosage for use in humans. Therapeutically effective dosages achieved in one animal model can be converted for use in another animal, including humans, using conversion factors known in the art (see, e.g., Freireich et al. (1966) Cancer Chemother. Reports, 50(4):219-244 and Table 2 for Equivalent Surface Area Dosage Factors).

TABLE 2

| | To: | | | | |
|---|---|---|---|---|---|
| | Mouse | Rat | Monkey | Dog | Human |
| From: | (20 g) | (150 g) | (3.5 kg) | (8 kg) | (60 kg) |
| Mouse | 1 | 1/2 | 1/4 | 1/6 | 1/12 |
| Rat | 2 | 1 | 1/2 | 1/4 | 1/7 |
| Monkey | 4 | 2 | 1 | 3/5 | 1/3 |
| Dog | 6 | 4 | 3/5 | 1 | 1/2 |
| Human | 12 | 7 | 3 | 2 | 1 |

4. Pharmaceutical Compositions, Methods of Administration, and Dosage

The disclosure provides pharmaceutical compositions comprising B7-H3 agents. Such compositions may be suitable for pharmaceutical use and administration to patients. The compositions typically comprise one or more antibodies of the present invention and a pharmaceutically acceptable excipient. The phrase “pharmaceutically acceptable excipient” includes any and all solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. The administration may, for example, be intravenous, intraperitoneal, intramuscular, intracavity, subcutaneous or transdermal. It may also be possible to obtain compositions which may be topically or orally administered, or which may be capable of transmission across mucous membranes.

Solutions or suspensions used for intradermal or subcutaneous application typically include one or more of the following components: a sterile diluent such as water for injection, saline solution, fixed oils, polyethylene glycols, glycerin, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and/or by the use of surfactants. Prolonged absorption of the injectable compositions can be brought about by including in the composition an agent which delays absorption, for example, aluminum monostearate, and gelatin.

Oral compositions generally include an inert diluent or an edible carrier. They can be enclosed in gelatin capsules or compressed into tablets. For oral administration, the antibodies can be combined with excipients and used in the form of tablets, troches, or capsules. Pharmaceutically compatible binding agents, and/or adjuvant materials can be included as part of the composition. The tablets, pills, capsules, troches, and the like can contain any of the following ingredients, or compounds of a similar nature; a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, detergents, bile salts, and fusidic acid derivatives. Transmucosal administration may be accomplished, for example, through the use of lozenges, nasal sprays, inhalers, or suppositories. For example, in case of antibodies and Ig fusion proteins that comprise the Fc portion, compositions may be capable of transmission across mucous membranes in intestine, mouth, or lungs (e.g., via the FcRn receptor-mediated pathway as described in U.S. Pat. No. 6,030,613). For transdermal administration, the active compounds may be formulated into ointments, salves, gels, or creams as generally known in the art. For administration by inhalation, the antibodies may be delivered in the form of an aerosol spray from pressured container or dispenser, which contains a suitable propellant, e.g., a gas such as carbon dioxide, or a nebulizer.

In certain embodiments, the presently disclosed B7-H3 agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions containing the presently disclosed antibodies can also be used as pharmaceutically acceptable carriers. These can be prepared according to methods known to those skilled in the art, for example, as described in U.S. Pat. No. 4,522,811.

It may be advantageous to formulate oral or parenteral compositions in a dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of the composition of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

For any composition used in the present invention, the therapeutically effective dose can be estimated initially from cell culture assays. Examples of suitable bioassays include DNA replication assays, cytokine release assays, transcription-based assays, binding assays, creatine kinase assays, assays based on the differentiation of pre-adipocytes, assays based on glucose uptake in adipocytes, immunological assays other assays as, for example, described in the Examples. The data obtained from the cell culture assays and animal studies can be used in formulating a range of dosage for use in humans. A dose may be formulated in animal models to achieve a circulating plasma concentration range that includes the IC₅₀ (i.e., the concentration of the therapeutic that achieves a half-maximal inhibition of symptoms). Circulating levels in plasma may be measured, for example, by high performance liquid chromatography. The effects of any particular dosage can be monitored by a suitable bioassay. The dosage lies preferably within a range of circulating concentrations with little or no toxicity. The dosage may vary depending upon the dosage form employed and the route of administration utilized.

The following Examples do not in any way limit the scope of the invention. One of ordinary skill in the art will recognize the numerous modifications and variations that may be performed without altering the spirit or scope of the present invention. Such modifications and variations are encompassed within the scope of the invention. The entire contents of all references, patents, and published patent applications cited throughout this application are herein incorporated by reference.

### EXAMPLES

### Example 1

### Isolation of Genomic DNA for B7-H3

B7-H3 RT-PCR was performed using oligonucleotides corresponding to the regions containing the initiation methionine and termination codons of human B7-H3 (Genbank accession No. AF302102) using the following PCR conditions.

PCR enzymes used in this study include KOD Hot Start (Novagen, Madison, Wis.), Advantage™ 2 (Clontech, Palo Alto, Calif.), and Platinum Taq (Invitrogen, Carlsbad, Calif.) enzymes according to manufacturer's protocols. When necessary, reaction conditions were supplemented to a final concentration of 1 M Betaine and 3% DMSO for robust amplification. Primers PW264 (SEQ ID NO:23) with imbedded attB1/Kozak and PW265 (SEQ ID NO:24) with imbedded attB2 sites, were used to amplify human B7-H3 coding sequences from first strand cDNA of spleen, lymph node, heart, liver, pancreas, and placenta as templates (Clontech). Human B7-H3 VCVC coding region sequences were obtained corresponding to sequences represented in existing database entries (Celera Human and Mouse Genomic Assemblies, Celera Genomics, Rockville, Md.): AX357960, AX097550, AX047072, AX097556, and AX136363 among others. Human B7-H3 VC form was constructed by deletion of human B7-H3 VCVC C₁-V₂ domains, matching the coding sequence of NM_{—}025240. PW270 (SEQ ID NO:26) and PW271 (SEQ ID NO:27) were used to amplify mouse B7-H3 sequences from mouse embryo first strand cDNA. PCR products with a size of 951 bp were subcloned, clearly revealing the correct splicing of the 7 predicted bona fide exons with a 100% accuracy, without inclusion of any pseudo-exons sequences in any analyzed clone. Mouse B7-H3 coding sequences corresponded to existing database entries BC019436, AX370312, and NM_{—}133983. Primers PW284 (SEQ ID NO:29) and PW267 (SEQ ID NO:25) were used to semi-quantitatively assess the relative contribution of human B7-H3 VC (690 bp) and VCVC (1344 bp) transcripts in cDNA panels (Clontech). Southern blots were performed by alkaline transfer of DNA onto Zetaprobe™ GT membrane (BioRad, Hercules, Calif.) and hybridized using ³²P end-labeled PW278 (SEQ ID NO:28) as detection oligonucleotide (Ling et al. (2001) J. Immunol., 166:7300-7308).

Monkey and hamster genomic DNAs were isolated from COS and CHO cell lines (Ling et al. (1999) Genomics 60:341-355). Genomic PCR was performed using PW358 (SEQ ID NO:30) and PW359 (SEQ ID NO:31) as primers based on nucleotides conserved between human and mouse B7-H3 sequences (amino acids 60-66, 216-221, and 278-284, 434-439 of SEQ ID NO:6). Amplification reactions were performed in duplicate and multiple subcloned products were analyzed by sequencing. Orientation of V-intron-C domains within monkey genomic DNA was determined by PCR using PW381 (SEQ ID NO:33) and PW384 (SEQ ID NO:34), and within hamster genomic DNA using PW358 (SEQ ID NO:30) and PW378 (SEQ ID NO:32).

DNA analysis was performed as follows. An aliquot (0.25-0.5 μg) of plasmid DNA was combined with 1 μl of 5 μM primer and 3 μl of 2 fold diluted ABI PRISM™ BigDye™ Terminator Cycle Sequencing Ready Reaction Kit mix (Version 3.0). The volumes were adjusted to 10 μl with 10 mM Tris-HCl (pH 8.0), and amplification reactions were performed on PTC-225 cycler (MJ Research, Waltham, Mass.) for 25 cycles (96° C. for 10 seconds, 50° C. for 5 seconds and 60° C. for 4 minutes). 10 μl of water was added to the reactions and the excess dye was removed by gel filtration on a 96-well Millipore filter plate with G-50 beads. The samples were heat-denatured for 2 min at 90-95° C. and electrophorezed on ABI3100 Genetic Analyzer (Applied Biosystems, Foster City, Calif.) under conditions recommended by the manufacturer. Manual sequence editing was performed using Sequencher™ 4.1 (Gene Codes, Ann Arbor, Mich.).

Sequence determination of the major amplification product resulted in a 1605 bp sequence distinct from B7-H3 and consistent with other EST and patent database entries. Whereas B7-H3 contained single V and C domain (B7-H3 VC form), the variant clones contained a duplicated V and C domains (B7-H3 VCVC form). To determine whether these clones were gene products independent from B7-H3, the genomic organization of the VCVC clones was analyzed by human genomic database query (Celera Genomics). The resulting match corresponded to one chromosome 15 genomic axis GA_x2HTBL4SSTP-04, suggesting a single genetic origin of both B7-H3 VC and B7-H3 VCVC variants. FIG. 1B depicts genomic organization of human and mouse B7-H3 gene loci. Assemblies are based on selected portions of Celera human genomic axis GA_x2HTBL4SSTP and Celera mouse genomic axis GA_x5J8B7W7NM9. Bars denote relative locations of Alu and SVA complex repeats, simple repeats, transcript exon structure, and domain name. The genomic organization of B7-H3 VCVC revealed 9 exons encoding leader domain, V₁ domain, C₁ domain, V₂ domain, C₂ domain, transmembrane domain and three cytoplasmic domains. Approximately 13.5 kb of genomic sequence separated initiation methionine on exon 1 from termination codon on exon 9 of the human B7-H3 locus. Exon delineation of human B7-H3 VC to the B7-H3 locus revealed alternative splicing from exon 2 (V₁) to exon 5 (C₂), resulting in the deletion of the C₁ and V₂ domains from this gene product.

Sequence analysis, alignments, and phylogenetic guide tree generation were performed using the Align module (Clustal W) of Vector NTI™ version 7.1 (Informax Inc, North Bethesda, Md.). Sequence alignment and phylogram analysis of primate sequences revealed a greater degree of sequence similarity between intraspecies V-intron-C DNA sequences than interspecies V-intron-C DNA sequences, i.e., human V₁-intron-C₁ is more similar to human V₂-intron-C₂ than monkey V-intron-C. Indeed, 100% identity was observed between the monkey C domain sequences analyzed. Intraspecies clustering is further supported by alignment of the non-coding intra-VC intron sequence where higher sequence conservation is observed than the duplicated V exons (97% vs. 94%). Although conserved nucleotides were found in intron sequences of all four species, no conserved nucleotide pairs were found to be shared exclusively between rodent and primate species, (data not shown) arguing against a common “pre-duplicated” primate VCVC molecule ancestral to both primates and rodents. Sequences flanking human B7-H3 V-intron-C domains revealed no such sequence conservation, suggesting the duplication in primates is very recent or the V-intron-C region is extremely protected from mutation. Thus, these data strongly support a model of multiple independent emergence of tandem VC repeats within human and monkey species.

### Example 2

### Relative Transcript Contribution Between B7-H3 Splice Variants

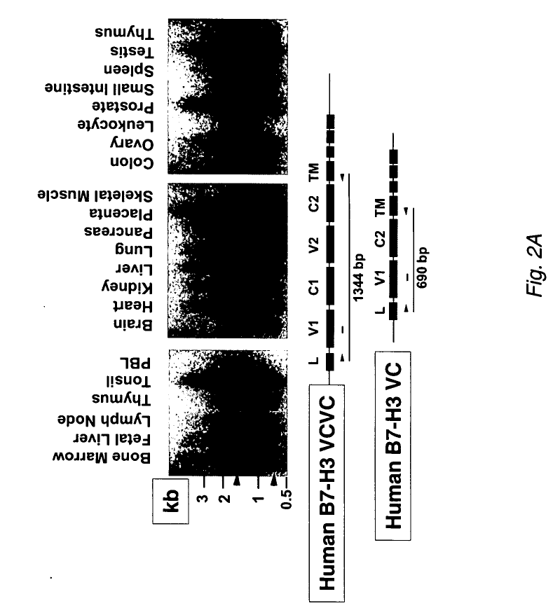

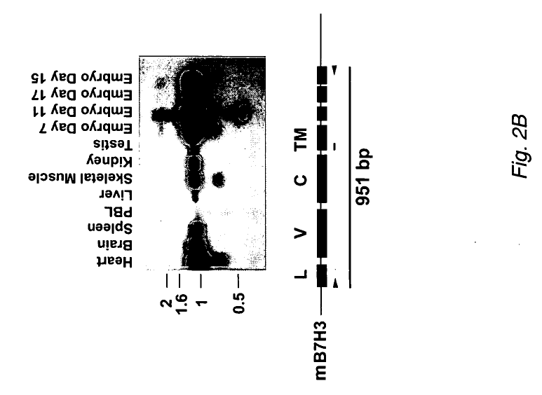

RT-PCR was performed on various human tissue samples followed by hybridization with radiolabeled oligonucleotide probes (FIG. 2A). Two unambiguous bands were detected with one migrating with a relative mobility corresponding to 1344 bp, consistent with the predicted size of the B7-H3 VCVC amplification product. A minor band was also detected corresponding to 690 bp, consistent with the predicted size of a VC amplification product. RT-PCR of three cDNA panels revealed this pattern in every tissue examined, except for leukocyte/PBL, where no amplification products were detected. Phosphoimage quantitation of hybridized regions indicated a ratio between 12.7:1 (brain) and 92.1:1 (kidney). In no case was the hybridization signal to the smaller B7-H3 VC product found to be greater than those seen for the B7-H3 VCVC product. Given that the amplification reactions favor smaller amplification products, the relative abundance of larger B7-H3 VCVC products over that of the smaller B7-H3 VC products suggest that B7-H3 VCVC is the dominant transcript species of naturally expressed human B7-H3 gene product in human tissues. The rarity of the B7-H3 VC form is consistent with the observed single 4.1 kb B7-H3 band in previously reported Northern blots, most probably representing the predominant B7-H3 VCVC product (Chapoval et al. (2001) Nat. Immunol., 2:269-274). In comparison, PCR-Southern blots of mouse B7-H3 show expression in all tissues examined with a dominant band at approximately 1 kb, consistent with the predicted 951 bp amplified product (FIG. 2B). Other B7-H3 hybridization signals were also detected in mouse embryo, heart and skeletal muscle tissues. Unlike primates, rodent sequences have only the single VC form, due to codon degeneration of putative C₁ and V₂ exons.

As demonstrated below, both human B7-H3 VC and VCVC forms have similar biological activity in vitro, suggesting that the tandemly duplicated exons are functionally equivalent in cell based assays. Such functional redundancy may explain the tolerance of C₁-V₂ exon loss without an adverse effect in physiology.

### Example 3

### Comparative Genomic Analysis of Mouse and Human B7-H3

Given that the segmental duplication of VC domains appears to be unique to B7-H3 amongst the B7-family of proteins, we next examined whether the sequence of B7-H3 was distinct from those of other costimulatory ligands based on codon base substitution. One method of determining the rates of molecular evolution is by the measurement of predicted mutation rates between synonymous and nonsynonymous sites within codons. Additional rodent versus primate sequence comparisons were performed for the V and C exons of other known ligands: B7-1, B7-2, GL50, PD-L1, and PD-L2. These ligands share structural similarities in which V and C domains reside in the extracellular portion of each molecule. The presence of shared structural motifs between these disparate molecules have propagated the notion that these molecules were derived from an ancestral sequence bearing V and C sequences. To determine the relative rates of divergence of V and C domains between mouse and humans, relative frequencies of nucleotide substitutions in synonymous and nonsynonymous codons were determined. Calculation of synonymous and nonsynonymous mutation frequencies were performed using Wisconsin Package GCG 10.0 Diverge module (GCG, Madison, Wis.). For B7-H3 alignments, exons corresponding to human V₁ and C₂ domains were aligned with mouse V and C domains. Of all the V and C domains examined, the ratio of synonymous substitutions vs. nonsynonymous mutations was less than 1 except for the V domain of B7-H3. The B7-H3 molecule had the lowest levels of synonymous mutations (d_{S}=0.129 substitutions per site) of all V domains while simultaneously also had the lowest levels of nonsynonymous mutations (d_{N}=0.026 substitutions per site) of all C domains. As a result, the B7-H3 molecule is distinct among costimulatory ligands in having the highest d_{N}:d_{S} ratio in the V domain while concurrently having the lowest d_{N}:d_{S} ratio in the C domain. The cross-domain divergence comparison between human V₂ and mouse V domain revealed a d_{S}=0.433 and d_{N}=0.037, while human C₁ and mouse C domains revealed a d_{S}=0.393 and d_{N}=0.034. The lower synonymous mutation rate seen between human V₁ and mouse V than human V₂ and mouse V implies that human V₁ and mouse V domains are orthologous. Assuming a linear mechanism of sequence evolution, dichotomy of nucleotide substitution rates between adjacent exons of the same molecule indicates that different selection processes occur between the V domains and C domains of B7-H3.

Based on mathematical models of molecular evolution, cases where synonymous substitution rates are greater than nonsynonymous substitution rates for a particular coding sequence reflects purifying selection. Purifying selection occurs when physiological constraints limits the levels of amino acid variation within the gene product. Purifying selection is evident in all B7-family V and C domains examined with the exception of the B7-H3 V₁ domain. For B7-H3 V₁, d_{S} is less than half that of B7-2 V and approximately one eighth that of PD-L1 V. Although B7-H3 V₁ d_{N} is low, it still exceeds that of B7-H3 V d_{S} for d_{N}:d_{S} ratio of 1.18. Cases in which d_{N}:d_{S} ratio is greater than 1 are unusual, and have been attributed to sequences undergoing positive selection for rapidly evolving function. It is also notable that the B7-H3 C₂ exon exhibits a d_{N}:d_{S} ratio of 0.063, the lowest of all exons examined, and is from one-fifth to one-fifteenth the levels seen for other C domains examined. Therefore, exons comprising B7-H3 were/are currently being actively maintained in a manner disparate to other costimulatory ligands.

### Example 4

### B7-H3 VC and VCVC Downregulate T Cell Activation

Based on the single VC unit observed in rodent B7-H3, we sought to determine whether the B7-H3 VC and VCVC forms found in humans had similar function in cell-based assay. The ability of B7-H3 VC and B7-H3 VCVC to downregulate T cell activation was observed for both proliferation and cytokine production levels. The experiments were conducted as follows.

Human B7-H3 VC or VCVC construct entry vectors were cloned into bicistronic retroviral vectors encoding IRES-GFP. The recipient destination retroviral vectors were originally derived from GFP-RV vector (Ranganath (1998) J. Immunol., 161.3822-3826) and modified for Gateway recombination using a attB1-ccdB-attB2 cassette (Invitrogen). Virus-containing supernatants were generated and used to infect CHO.HLA-DR2 cells as previously described (Carter et al. (2002) Eur. J. Immunol., 32:634-643). CHO.HLA-DR2.B7-H3 VC and CHO.HLA-DR2.B7-H3 VCVC were selected by cell sorting based on GFP expression. A CHO.HLA-DR2 transfectant expressing similar GFP levels was used as a control in cellular assays. Generation of CHO.HLA-DR2.B7.1 and CHO.HLA-DR2.B7.2 has been described previously (Anderson et al. (2000) Nature Medicine, 6(2):211-214). CHO.HLA-DR2 transfectants were fixed in 0.2% paraformaldehyde at room temperature (RT) for 4 min and fixation quenched in 1 M lysine at RT for 4 min. Cells were washed once with PBS, resuspended in culture media (RPMI1640, 10% FCS) and used as antigen presenting cells in T cell proliferation assays.

Human CD4⁺ T cells were purified by negative selection from peripheral lymphocytes as described previously (Blair et al. (1998) J. Immunol., 160:12-15). CD4⁺ T cells (10⁵ cells/well) were cultured in flat-bottom 96-well plates with paraformaldehyde-fixed CHO.HLA-DR2 transfectants (1.25×10⁴ cells/well) in the presence of soluble anti-CD3 antibody (1 μg/ml, UCHT1, Pharmingen, San Diego, Calif.) and various concentrations of soluble anti-CD28 antibody (CD28.2, Pharmingen). Proliferation was determined by pulsing cultures with 1 Ci [³H]-thymidine per well for the last 5-12 hours of a 72-hour incubation period. To measure cytokine production, supernatants were harvested at 72 hours, and samples assayed by multiplex ELISA screening (Pierce Boston, Woburn, Mass.).

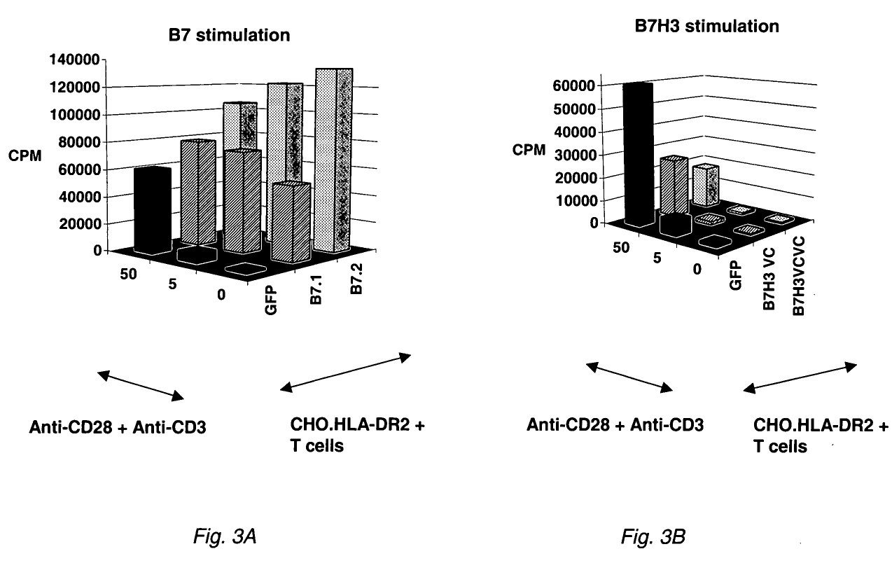

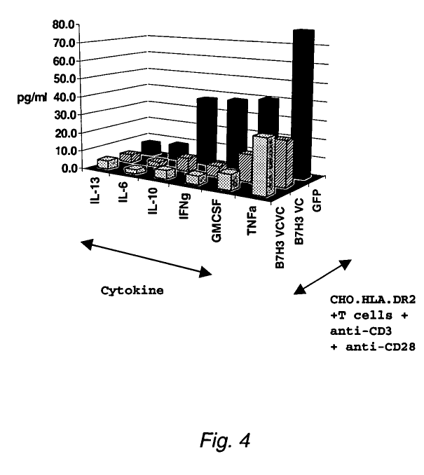

In order to determine functional activity of the B7-H3 VC and VCVC forms, the following cell-based assays were conducted. Purified human CD4⁺ T cells were stimulated with paraformaldehyde-treated CHO.HLA-DR2 transfectants in the presence of constant amounts of soluble anti-CD3 antibody and increasing concentrations of soluble anti-CD28 antibody. Activation of purified T cells with anti-CD3 in the presence of CHO.HLA-DR2-GFP transfectants resulted in no proliferation; proliferation levels were enhanced in the presence of anti-CD3 and anti-CD28 antibodies (FIGS. 3A-3B); stimulation of T cells with anti-CD3 antibody in the presence of CHO.HLA-DR2.B7-1 or B7-2 resulted in proliferation above the levels obtained with CHO.HLA-DR2-GFP control cells (FIG. 3A). Soluble anti-CD28 antibody enhanced the GFP control, but not the B7-1 and B7-2, responses. In contrast, stimulation of T cells in the presence of B7-H3 VC or B7-H3 VCVC led to decreased proliferative responses (FIG. 3B). With anti-CD3 (1 g/ml) and low costimulation (5 ng/ml of anti-CD28 antibody), cytokine production was significantly reduced upon B7-H3 VC and B7-H3 VCVC stimulation. IL-10 (˜81%), TNF-α (˜69%), IFN-γ (˜85%), and GM-CSF (˜65%) levels were dramatically reduced in cultures stimulated with either B7-H3 VC or VCVC cultures relative to GFP controls (FIG. 4). Negligible amounts of IL-1A, IL-2, IL-4, IL-6, and IL-13 were detected in these assay conditions. These findings indicate that neither B7-H3 VC nor B7-H3 VCVC function as a costimulatory molecule and suggest that B7-H3 VC and B7-H3 VCVC cell surface molecules engage receptors on T cells that serve as negative regulators of activation. Addition of anti-CD28 antibody at concentrations as high as 200 ng/ml could only partially rescue the B7-H3 inhibitory effect on proliferation.

### Example 5

### B7-H3 VC and VCVC Downregulate T Cell Activation

To further characterize B7-H3 function, experiments were performed on a single (CIS) or a separate (TRANS) surface to determine whether B7-H3 downregulation of T cell responses requires coordinate TCR/B7-H3 receptor engagement. CIS beads contained anti-CD3 antibodies and purified fusion proteins B7-H3 VC-Ig or B7-H3 VCVC-Ig, whereas TRANS beads contained either anti-CD3 antibody and B7-H3 VC-Ig or B7-H3 VCVC-Ig.

Bead stimulation of T cells was performed as follows. Anti-CD3 (UCHT1, Pharmigen), human B7-H3 VC-Ig, human B7-H3 VCVC-Ig and control Ig were covalently attached to polyurethane-coated tosyl-activated Dynabeads (Dynal, Lake Success, N.Y.). Beads were prepared with a constant sub-optimal anti-CD3 antibody concentration (1 μg, 20% of the total bound protein) and B7-H3-Ig or control Ig (4 μg, 80% of total bound protein (Bennett et al. (2003) J. Immunol., 170:711-718). Beads have a binding capacity of 5 μg per 10⁷ beads. CIS beads contain both anti-CD3 and B7-H3 on the same bead, TRANS beads consist of two types of beads, one containing anti-CD3 antibody and the other containing B7-H3-Ig (Bennett et al. (2003) J. Immunol., 170:711-718). To maintain equal bead-to-cell ratios under CIS and TRANS conditions, beads coated with control IgG were added to CIS cultures. Protein-coated beads were added to purified CD4⁺ T cells (10⁵ cells/well) in flat bottomed 96-well microtiter plates at a ratio of 1:1. Proliferation was determined by pulsing cultures with 1 Ci [³H]-thymidine per well for the last 6-16 hour of a 72-hour incubation period.

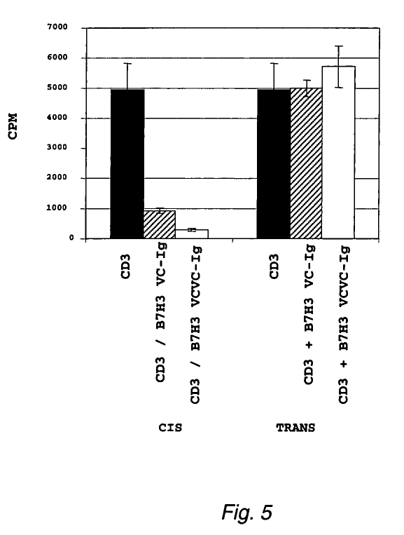

As shown in FIG. 5, inhibition of proliferation was only observed when cells were activated with CIS beads. Altogether, these findings suggest that the B7-H3 receptor and the TCR need to be in close proximity for the downregulation of T cell activation. These data suggest that for the B7-H3 receptor pathway to modulate a T cell response, both activating and inhibitory signals must emanate from the same cell.

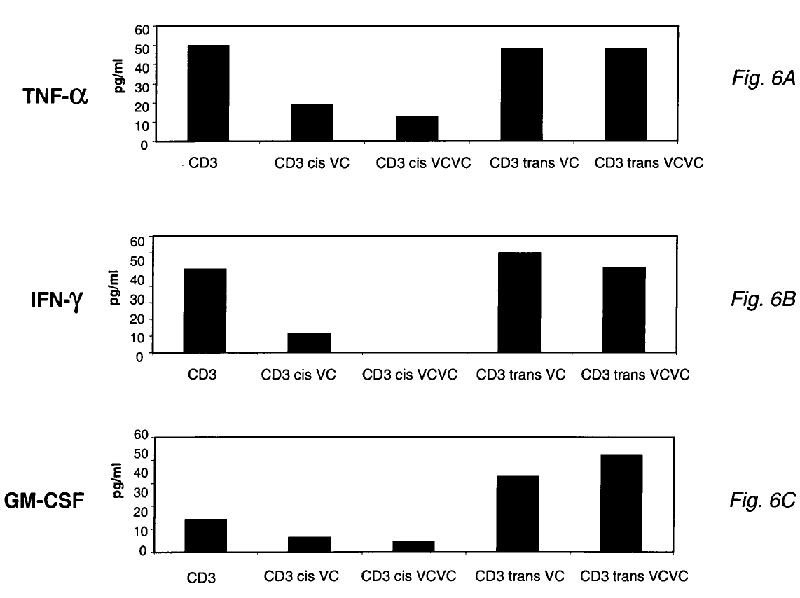

The amount of cytokines in the supernatants was measured at 72 hours using multiplex ELISA screening: TNF-α (FIG. 6A), IFN-γ (FIG. 6B), and GM-CSF (FIG. 6C). The ability of B7-H3 VC and B7-H3 VCVC to downregulate T cell activation was assessed by cytokine production levels. With anti-CD3 (1 g/ml) and low costimulation (5 ng/ml anti-CD28 antibody), cytokine production was significantly reduced upon B7-H3 VC and B7-H3 VCVC stimulation (FIGS. 6A-6C). IL-10 (˜81%), TNF-α (˜69%), IFN-γ (˜85%), and GM-CSF (˜65%) levels were dramatically reduced in cultures stimulated with either B7-H3 VC or VCVC cultures relative to GFP controls. Negligible amounts of IL-1A, IL-2, IL-4, IL-6, and IL-13 were detected in these assay conditions. These findings indicate that neither B7-H3 VC nor B7-H3 VCVC function as a costimulatory molecule and suggest that B7-H3 VC and B7-H3 VCVC cell surface molecules engage receptors on T-cells that serve as negative regulators of activation.

Results show that TCR/B7H3 (VC or VCVC) activation of T cells leads to downregulation of T cell responses. Proliferation and cytokine production is decreased in TCR/B7-H3 activated T cells relative to cells activated by TCR alone. This data suggests that engagement of the B7-H3 receptor on T cell delivers a negative signal. Results also suggest that physical proximity between TCR and the B7-H3 receptor may be required in order to downregulate T cell activation via the B7-H3 receptor.

The ability of B7-H3 VC and VCVC to downregulate CD4⁺ T-cell activation is reminiscent of negative signals produced by engagement of CTLA4 by either of B7-proteins or by PD-1 by either of the PD-L1 and PD-L2 proteins. Furthermore, the experiments with B7-H3 coupled to a solid matrix indicates that both TCR and B7-H3 receptor signals are delivered by the same cell. Similar requirements have been described for negative signaling by either CTLA-4 or PD-1 (Griffin et al. (2000) J. Immunol., 164:4433; and Bennett et al. (2003) J. Immunol., 170:711-718). Finally, both human B7-H3 VC and human B7-H3 VCVC molecules appear to be redundant in their ability to modulate CD4 T-cell responses.

### Example 6

### Therapeutic Efficacy in Psoriasis Patients

Modulation of immune response regulated by B7-H3 is useful in instances where an immunosuppressive effect or augmentation of immune response is desired. B7-H3 agonists (e.g., soluble forms of B7-H3) may be used to prevent and/or to reduce severity and/or symptoms of diseases or conditions that involve an aberrantly elevated immune response, including response to self antigens as, for example, in autoimmune disorders. B7-H3 antagonists, on the other hand, may be administered to subjects having an undesirably low immune response as for example it may occur in cancers or immunosuppressive disorders.

Psoriasis is considered to a typical T-cell-mediated autoimmune disease. Psoriasis is a chronic inflammatory skin disease mediated, in part, through IFN-γ production by activated lesional T cells (Th₁ skewed).

Most commonly, soluble proteins are administered in an outpatient setting by weekly administration at 0.1-10 mg/kg dose by slow intravenous (IV) infusion. The appropriate therapeutically effective dose of an antagonist is selected by a treating clinician and would range approximately from 1 μg/kg to 20 mg/kg, from 1 μg/kg to 10 mg/kg, from 1 μg/kg to 1 mg/kg, from 10 μg/kg to 1 mg/kg, from 10 μg/kg to 100 μg/kg, from 100 μg/kg to 1 mg/kg, and from 500 μg/kg to 5 mg/kg.

To evaluate the effects on skin T cells, an antibody against B7-H3, an antibody against a B7-H3 receptor, or B7-H3-Ig is administered for 12 consecutive weeks to randomized groups of psoriasis patients with normalized disease severity (minimum PASI (Psoriasis Activity and Severity Index) score of 12). To assess clinical improvement in patients over time and to monitor their response to therapy, the PASI scoring system can be used (Fredriksson et al. (1978) Dermatologica, 157:238-244; and Marks et al. (1989) Arch. Dermatol., 1989; 125:235-240).

In brief, elements of the PASI score include:(1) body regions affected as percent of body surface area (BSA); (2) extent to which body region is affected (on a scale of 1-10); and (3) extent of psoriatic changes (erythema, infiltration, desquamation) on a scale of 0-4). PASI score is calculated as follows: ((0.1×(erythema head)+(infiltration head)+(desquamation head))×(extent of head affected))+((0.2×((erythema trunk)+(infiltration trunk)+(desquamation trunk))×(extent of trunk affected))+((0.3×((erythema upper extremities)+(infiltration upper extremities)+(desquamation upper extremities))×(extent of upper extremities affected))+((0.4×((erythema lower extremities)+(infiltration lower extremities)+(desquamation lower extremities))×(extent of lower extremities affected)). The minimum score is 0, while the maximum score=72. A reduction in PASI score is indicative of the effective treatment. It is anticipated that at least 50% of individuals receiving the treatment would exhibit a reduction in PASI score and an improvement in their condition.

The specification is most thoroughly understood in light of the teachings of the references cited within the specification which are hereby incorporated by reference. The embodiments within the specification provide an illustration of embodiments of the invention and should not be construed to limit the scope of the invention. The skilled artisan readily recognizes that many other embodiments are encompassed by the invention. All publications and patents cited and sequences identified by accession or database reference numbers in this disclosure are incorporated by reference in their entirety. To the extent the material incorporated by reference contradicts or is inconsistent with the present specification, the present specification will supercede any such material. The citation of any references herein is as not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may very depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

1. A method of inhibiting activation of a lymphocyte, the method comprising contacting the lymphocyte with a B7-H3 agonist and allowing the agonist to inhibit the activation of the lymphocyte.

2. The method as in claim 1, wherein the B7-H3 agonist is a soluble form of B7-H3.

3. The method as in claim 3, wherein the B7-H3 agonist comprises SEQ ID NO:15.

4. The method as in claim 2, wherein the soluble form comprises at least one V domain of B7-H3.

5. The method as in claim 4, wherein the V domain comprises: (a) SEQ ID NO:7 or (b) an amino acid sequence which is substantially identical to SEQ ID NO:7.

6. The method as in claim 4, wherein the soluble form of B7-H3 further comprises at least one C domain of B7-H3.

7. The method as in claim 4, wherein the soluble form of B7-H3 further comprises an Fc region of an antibody.

8. The method as in claim 7, wherein the soluble form of B7-H3 comprises: (a) an amino acid sequence chosen from SEQ ID NO:16, SEQ ID NO:17, SEQ ID NO:18, SEQ ID NO:19, SEQ ID NO:20, SEQ ID NO:21, or SEQ ID NO:22; or (b) an amino acid sequence which is substantially identical to at least one of the sequences chosen from SEQ ID NO:16, SEQ ID NO:17, SEQ ID NO:18, SEQ ID NO:19, SEQ ID NO:20, SEQ ID NO:21, or SEQ ID NO:22.

9. The method as in claim 7, wherein the soluble from of B7-H3 comprises: (a) an amino acid sequence chosen from SEQ ID NO:10, SEQ ID NO:12, or SEQ ID NO:14; or (b) an amino acid sequence which is substantially identical to at least one of the sequences chosen from SEQ ID NO:10, SEQ ID NO:12, or SEQ ID NO:14.

10. The method as in claim 3, wherein the B7-H3 agonist is coupled with a primary stimulatory molecule.

11. The method as in claim 10, wherein the soluble form of B7-H3 and the primary stimulatory molecule are spaced by no more than 100 μm.

12. The method as in claim 1, wherein the B7-H3 antagonist is a nucleic acid encoding amino acid of SEQ ID NO:15.

13. A method of enhancing activation of a lymphocyte, the method comprising contacting the lymphocyte with a B7-H3 antagonist and allowing the antagonist to enhance the activation of the lymphocyte.

14. The method as in claim 13, wherein the lymphocyte is human.

15. The method as in claim 13, wherein the B7-H3 antagonist is an antibody to B7-H3 or an antibody against a B7-H3 receptor.

16. The method as in claim 13, wherein the B7-H3 antagonist is an antisense nucleic acid or a siRNA.

17. The method as any one of claims 1 or 13, wherein the lymphocyte is a T cell.

18. The method as in claims 17, wherein the T cell is a CD4⁺ T cell.

19. The method as any one of claims 1 or 13, wherein the lymphocyte is in a mammal.

20. The method as in claim 19, wherein the mammal is afflicted with or is at risk for at least one of: an immunologic disorder, a cancer, or an infectious disease.

21. The method as in claim 19, wherein the mammal is treated with Factor VIII or Factor IX.


SEQUENCE LISTING (34 sequences)

SEQ ID NO: 1 - DNA, 951 bp, Homo sapiens
atgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggccctgtgggtgaccgtggggctgtctgtctgtctcattgcactgctggtggccctggctttcgtgtgctggagaaagatcaaacagagctgtgaggaggagaatgcaggagctgaggaccaggatggggagggagaaggctccaagacagccctgcagcctctgaaacactctgacagcaaagaagatgatggacaagaaatagcctga

SEQ ID NO: 2 - protein, 316 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 3 - DNA, 951 bp, Mouse
atgcttcgaggatggggtggccccagtgtgggtgtgtgtgtgcgcacagcactgggggtgctgtgcctctgcctcacaggagctgtggaagtccaggtctctgaagaccccgtggtggccctggtggacacggatgccaccctacgctgctccttttccccagagcctggcttcagtctggcacagctcaacctcatctggcagctgacagacaccaaacagctggtgcacagcttcacggagggccgggaccaaggcagtgcctactccaaccgcacagcgctcttccctgacctgttggtgcaaggcaatgcgtccttgaggctgcagcgcgtccgagtaaccgacgagggcagctacacctgctttgtgagcatccaggactttgacagcgctgctgttagcctgcaggtggccgccccctactcgaagcccagcatgaccctggagcccaacaaggacctacgtccagggaacatggtgaccatcacgtgctctagctaccagggctatccggaggccgaggtgttctggaaggatggacagggagtgcccttgactggcaatgtgaccacatcccagatggccaacgagcggggcttgttcgatgttcacagcgtgctgagggtggtgctgggtgctaacggcacctacagctgcctggtacgcaacccggtgttgcagcaagatgctcacggctcagtcaccatcacagggcagcccctgacattcccccctgaggctctgtgggtaaccgtggggctctctgtctgtcttgtggtactactggtggccctggctttcgtgtgctggagaaagatcaagcagagctgcgaggaggagaatgcaggtgccgaggaccaggatggagatggagaaggatccaagacagctctacggcctctgaaaccctctgaaaacaaagaagatgacggacaagaaattgcttga

SEQ ID NO: 4 - protein, 316 aa, Mouse
MLRGWGGPSVGVCVRTALGVLCLCLTGAVEVQVSEDPVVALVDTDATLRCSFSPEPGFSLAQLNLIWQLTDTKQLVHSFTEGRDQGSAYSNRTALFPDLLVQGNASLRLQRVRVTDEGSYTCFVSIQDFDSAAVSLQVAAPYSKPSMTLEPNKDLRPGNMVTITCSSYQGYPEAEVFWKDGQGVPLTGNVTTSQMANERGLFDVHSVLRVVLGANGTYSCLVRNPVLQQDAHGSVTITGQPLTFPPEALWVTVGLSVCLVVLLVALAFVCWRKIKQSCEEENAGAEDQDGDGEGSKTALRPLKPSENKEDDGQEIA

SEQ ID NO: 5 - DNA, 1605 bp, Homo sapiens
atgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggccctgtgggtgaccgtggggctgtctgtctgtctcattgcactgctggtggccctggctttcgtgtgctggagaaagatcaaacagagctgtgaggaggagaatgcaggagctgaggaccaggatggggagggagaaggctccaagacagccctgcagcctctgaaacactctgacagcaaagaagatgatggacaagaaatagcctga

SEQ ID NO: 6 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 7 - protein, 112 aa, Homo sapience
ALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVA

SEQ ID NO: 8 - DNA, 1670 bp, Chimera
atgggggtactgctcacacagaggacgctgctcagtctggtccttgcactcctgtttccaagcatggccagcatgctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggcagggtcggggtccgagccccgcggaccgacaatcaagccctgtcctccatgcaaatgcccaggtaagtcactagaccagagctccactcccgggagaatggtaagtgctataaacatccctgcactagaggataagccatgtacagatccatttccatctctcctcatcagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggctttcgcatgcgccgtcaacaacaaagacctcccagcgcccatcgagagaaccatctcaaaacccaaaggtgagagctgcagcctgactgcatgggggctgggatgggcataaggataaaggtctgtgtggacagccttctgcttcagccatgacctttgtgtatgtttctaccctcacagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 9 - protein, 482 aa, Chimera
MGVLLTQRTLLSLVLALLFPSMASMLEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEAGSGSEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 10 - DNA, 2324 bp, Chimera
atgggggtactgctcacacagaggacgctgctcagtctggtccttgcactcctgtttccaagcatggccagcatgctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggcagggtcggggtccgagccccgcggaccgacaatcaagccctgtcctccatgcaaatgcccaggtaagtcactagaccagagctccactcccgggagaatggtaagtgctataaacatccctgcactagaggataagccatgtacagatccatttccatctctcctcatcagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggctttcgcatgcgccgtcaacaacaaagacctcccagcgcccatcgagagaaccatctcaaaacccaaaggtgagagctgcagcctgactgcatgggggctgggatgggcataaggataaaggtctgtgtggacagccttctgcttcagccatgacctttgtgtatgtttctaccctcacagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 11 - protein, 700 aa, Chimera
MGVLLTQRTLLSLVLALLFPSMASMLEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEAGSGSEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 12 - DNA, 1670 bp, Mouse
atgggggtactgctcacacagaggacgctgctcagtctggtccttgcactcctgtttccaagcatggccagcatggtggaagtccaggtctctgaagaccccgtggtggccctggtggacacggatgccaccctacgctgctccttttccccagagcctggcttcagtctggcacagctcaacctcatctggcagctgacagacaccaaacagctggtgcacagcttcacggagggccgggaccaaggcagtgcctactccaaccgcacagcgctcttccctgacctgttggtgcaaggcaatgcgtccttgaggctgcagcgcgtccgagtaaccgacgagggcagctacacctgctttgtgagcattcaggactttgacagcgctgctgttagcctgcaggtggccgccccctactcgaagcccagcatgaccctggagcccaacaaggacctacgtccagggaacatggtgaccatcacgtgctctagctaccagggctatccggaggccgaggtgttctggaaggatggacagggagtgcccttgactggcaatgtgaccacatcccagatggccaacgagcggggcttgttcgatgttcacagcgtgctgagggtggtgctgggtgctaacggcacctacagctgcctggtacgcaacccggtgttgcagcaagatgctcacggctcagtcaccatcacagggcagcccctgacattcccccctgaggcagggtcggggtccgagccccgcggaccgacaatcaagccctgtcctccatgcaaatgcccaggtaagtcactagaccagagctccactcccgggagaatggtaagtgctataaacatccctgcactagaggataagccatgtacagatccatttccatctctcctcatcagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggctttcgcatgcgccgtcaacaacaaagacctcccagcgcccatcgagagaaccatctcaaaacccaaaggtgagagctgcagcctgactgcatgggggctgggatgggcataaggataaaggtctgtgtggacagccttctgcttcagccatgacctttgtgtatgtttctaccctcacagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 13 - protein, 482 aa, Mouse
MGVLLTQRTLLSLVLALLFPSMASMVEVQVSEDPVVALVDTDATLRCSFSPEPGFSLAQLNLIWQLTDTKQLVHSFTEGRDQGSAYSNRTALFPDLLVQGNASLRLQRVRVTDEGSYTCFVSIQDFDSAAVSLQVAAPYSKPSMTLEPNKDLRPGNMVTITCSSYQGYPEAEVFWKDGQGVPLTGNVTTSQMANERGLFDVHSVLRVVLGANGTYSCLVRNPVLQQDAHGSVTITGQPLTFPPEAGSGSEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 14 - protein, 112 aa, Artificial sequence
  Conserved regions in mammalian B7-H3 V1 exon
XXXVQVXXXXXXXXXXXDATLCCSFXXEPGFSLAQLNLIWQLTDTKQLVHSXXXXXXXXXXXANRTXXXXXXXXXXXXXLRLQRVXXXXXXXXXXXXXXXXXGSAAXSLQVA

SEQ ID NO: 15 - protein, 3 aa, Artificial sequence
  Conserved amoung mammals
VQV

SEQ ID NO: 16 - protein, 8 aa, Artificial sequence
  Conserved among mammals
DATLCCSF

SEQ ID NO: 17 - protein, 24 aa, Artificial sequence
  Conserved among mammals
EPGFSLAQLNLIWQLTDTKQLVHS

SEQ ID NO: 18 - protein, 4 aa, Artificial sequence
  Conserved among mammals
ANRT

SEQ ID NO: 19 - protein, 6 aa, Artificial sequence
  Conserved among mammals
LRLQRV

SEQ ID NO: 20 - protein, 4 aa, Artificial sequence
  Conserved among mammals
GSAA

SEQ ID NO: 21 - protein, 5 aa, Artificial sequence
  Conserved among mammals
SLQVA

SEQ ID NO: 22 - protein, 112 aa, Artificial sequence
  Conserved amino acids in human B7-H3 V1 and V2
AXEVQVPEDPVVALVGTDATLXCSFSPEPGFSLAQLNLIWQLTDTKQLVHSFXEGXDQGSAYANRTALFPDLLAQGNASLRLQRVRVADEGSFTCFVSIRDFGSAAVSLQVA

SEQ ID NO: 23 - DNA, 55 bp, Artificial sequence
  Primer
ggggacaagtttgtacaaaaaagcaggctccaccatgctgcgtcggcggggcagc

SEQ ID NO: 24 - DNA, 50 bp, Artificial sequence
  Primer
ggggaccactttgtacaagaaagctgggttcaggctatttcttgtccatc

SEQ ID NO: 25 - DNA, 22 bp, Artificial sequence
  Primer
ctctggggggaatgtcataggc

SEQ ID NO: 26 - DNA, 55 bp, Artificial sequence
  Primer
ggggacaagtttgtacaaaaaagcaggctccaccatgcttcgaggatggggtggc

SEQ ID NO: 27 - DNA, 50 bp, Artificial sequence
  Primer
ggggaccactttgtacaagaaagctgggttcaagcaatttcttgtccgtc

SEQ ID NO: 28 - DNA, 21 bp, Artificial sequence
  Primer
agctttgctgagggccaggac

SEQ ID NO: 29 - DNA, 22 bp, Artificial sequence
  Primer
ctgggagcactgtggttctgcc

SEQ ID NO: 30 - DNA, 21 bp, Artificial sequence
  Primer
ctggcacagctcaacctcatc

SEQ ID NO: 31 - DNA, 20 bp, Artificial sequence
  Primer
accaggcagctgtaggtgcc

SEQ ID NO: 32 - DNA, 22 bp, Artificial sequence
  Primer
ctgtgatggtgactgagccgtg

SEQ ID NO: 33 - DNA, 21 bp, Artificial sequence
  Primer
cgcgtgcgtgtggcggatgag

SEQ ID NO: 34 - DNA, 22 bp, Artificial sequence
  Primer
tacaggaatcagcactgggttc